SEQUENCE LISTING (16 sequences)

SEQ ID NO: 1 - protein, 174 aa, Homo sapiens
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 2 - DNA, 63 bp, Saccharomyces cerevisiae
atgaaattgaaaactgttagatctgctgttttgtcttctttgtttgcttctcaagttttgggt

SEQ ID NO: 3 - DNA, 126 bp, Artificial Sequence
  leader sequence
caaccaattgatgatactgaatctcaaactacttctgttaatttgatggctgatgatactgaatctgcttttgctactcaaactaattctggtggtttggatgttgttggtttgatatcgatggcc

SEQ ID NO: 4 - DNA, 522 bp, Artificial Sequence
  DNA encoding G-CSF copy 1 in the single chain G-CSF dimer
actccattgggtccagcttcttctttgccacaatcttttttgttgaaatgtttggaacaagttagaaaaattcaaggtgatggtgctgctttgcaagaaaaattgtgtgctacttataaattgtgtcatccagaagaattggttttgttgggtcattctttgggtattccatgggctccattgtcttcttgtccatctcaagctttgcaattggctggttgtttgtctcaattgcattctggtttgtttttgtatcaaggtttgttgcaagctttggaaggtatttctccagaattgggtccaactttggatactttgcaattggatgttgctgattttgctactactatttggcaacaaatggaagaattgggtatggctccagctttgcaaccaactcaaggtgctatgccagcttttgcttctgcttttcaaagaagagctggtggtgttttggttgcttctcatttgcaatcttttttggaagtttcttatagagttttgagacatttggctcaacca

SEQ ID NO: 5 - DNA, 531 bp, Artificial Sequence
  DNA encoding G-CSF copy 2 in the single chain G-CSF dimer
acccctctgggcccggccagcagtctgcctcagagttttttactgaaatgcttagaacaggtgcgtaaaatccagggcgatggcgcggccctgcaggaaaaactgtgcgcgacctataaactgtgccatcctgaagaactggtcctgttaggccatagcttaggcatcccgtgggcgcctctgagtagctgcccgagtcaggccctgcagctggccggctgcctgagtcagttacatagtggcttatttttatatcagggcttactgcaggcgttagaaggcattagtccggaactgggcccgaccctggataccttacagttagatgtcgcggattttgccaccaccatttggcagcagatggaagaattaggcatggcgcctgcgttacagcctacccagggcgccatgcctgcgtttgcgagtgcgtttcagcgtcgcgccggcggcgtgttagtggccagccatctgcagagctttctggaagtgagttatcgtgtgttacgccatctggcccagccttaatctaga

SEQ ID NO: 6 - protein, 348 aa, Artificial Sequence
  Single chain G-CSF dimer polypeptide
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 7 - DNA, 90 bp, Homo sapiens
atggctggacctgccacccagagccccatgaagctgatggccctgcagctgctgctgtggcacagtgcactctggacagtgcaggaagcc

SEQ ID NO: 8 - DNA, 522 bp, Artificial Sequence; identical to SEQ ID NO: 4
  DNA encoding single-chain G-CSF copy 1 (codon usage optimized for expression in CHO cells)

SEQ ID NO: 9 - protein, 6 aa, Artificial Sequence
  tag
HHHHHH

SEQ ID NO: 10 - protein, 8 aa, Artificial Sequence
  tag
MKHHHHHH

SEQ ID NO: 11 - protein, 10 aa, Artificial Sequence
  tag
MKHHAHHQHH

SEQ ID NO: 12 - protein, 14 aa, Artificial Sequence
  tag
MKHQHQHQHQHQHQ

SEQ ID NO: 13 - protein, 15 aa, Artificial Sequence
  tag
MKHQHQHQHQHQHQQ

SEQ ID NO: 14 - protein, 10 aa, Artificial Sequence
  tag
EQKLISEEDL

SEQ ID NO: 15 - protein, 8 aa, Artificial Sequence
  tag
DYKDDDDK

SEQ ID NO: 16 - protein, 9 aa, Artificial Sequence
  tag
YPYDVPDYA

(19) United States
(12) Patent Application Publication
(10) Pub. No.: US 2002/0142964 A1
(43) Pub. Date: Oct. 3, 2002

(54) Single-chain polypeptides

(76) Inventors: Torben Lauesgaard Nissen, Frederiksberg C (DK); Anne Dam Jensen, Copenhagen (DK)

Correspondence Address: Joanne Petithory, Maxygen, Inc., 515 Galveston Drive, Redwood City, CA, 94063, US

(21) Appl. No.: 10/003,496

(22) Filed: Nov. 1, 2001

Related U.S. Application Data
(60) Provisional application No. 60/245,727, filed on Nov. 2, 2000.

(51) Int. Cl. A61K 38/22; C07K 14/575

(52) U.S. Cl. 514/12; 530/399

(57) ABSTRACT

The invention relates to single-chain multimeric polypeptides comprising at least two units of a monomeric polypeptide linked via a peptide bond or a peptide linker, wherein the monomeric polypeptide is of a type that is biologically active in monomeric form, and to polypeptide conjugates having at least one non-polypeptide moiety covalently bound to an attachment group of the polypeptide. The polypeptide is preferably a G-CSF dimer bound to a polymer molecule, preferably to one or more polyethylene glycol molecules.

### CROSS-REFERENCE TO RELATED APPLICATIONS

Pursuant to 35 U.S.C. §119(e), this application claims priority to and benefit of U.S. Provisional Patent Application Serial No. 60/245,727 filed on Nov. 2, 2000, the disclosure of which is incorporated herein in its entirety for all purposes.

### COPYRIGHT NOTIFICATION

Pursuant to 37 C.F.R. §1.71(e), Applicants note that a portion of this disclosure contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

The present invention relates to single-chain multimeric polypeptides and polypeptide conjugates, in particular to multimeric cytokine polypeptides comprising at least two monomeric units of a cytokine polypeptide of a type that normally is biologically active in monomeric form.

### BACKGROUND OF THE INVENTION

It is well known that biopharmaceuticals such as granulocyte colony stimulating factor, erythropoietin, interferons, interleukins, growth hormones and factors and coagulation factors mediate their effect on cells by binding to specific receptors on cell surfaces, whereby cytokine-receptor complexes are formed that activate certain signal transduction pathways in the cells. The cytokines bind to their receptors as monomers or as multimers, e.g. dimers, trimers or tetramers.

Many cytokine pharmaceuticals and other injected protein pharmaceuticals suffer from the problem of an insufficient plasma half-life in the body, so that injections must be made relatively frequently in order to maintain a sufficient level of the protein in vivo. This is disadvantageous since it is both inconvenient for the patient and costly, and it is therefore often desirable to be able to increase the plasma half-life of protein pharmaceuticals. Various approaches have been used with the aim of increasing the plasma half-life of such proteins. Since it is known that increasing the size of the protein generally tends to increase the plasma half-life, several of these approaches have involved increasing the molecular weight by various means.

One such approach has been to increase the molecular weight by formation of polypeptide conjugates, for example by conjugation with a polymer such as polyethylene glycol (PEG). This process, called “PEGylation”, has been applied to a wide variety of proteins and has the advantage of often resulting in a reduced antigenicity. PEGylation has in some cases, however, led to a reduction in the biological activity of the PEGylated molecule.

Another approach for increasing the molecular weight of a protein is to link two or more proteins together either via a chemical linkage or via a peptide bond or peptide linker. This has most often been applied to conjugates of two different proteins, e.g. a protein of interest fused to human albumin or another abundant plasma protein. Further, there are a number of disclosures of various single-chain antibody constructs or single-chain multimeric antigen constructs used for vaccine purposes.

Certain single-chain polypeptide variants of cytokines that are biologically active in vivo only in dimeric form are disclosed in the literature.

Li et al. (J. Biol. Chem. 271(4): 1817-20, Jan. 1996; and J. Biol. Chem. 271(49): 31729-31734, Dec. 1996) disclose single-chain interleukin 5 (scIL-5) and mutants thereof produced in order to study receptor binding. Human interleukin 5 is biologically active as a disulfide-linked homodimer, and the scIL-5 mutants of Li et al. were based on scIL-5 constructed by linking two IL-5 monomers with a Gly-Gly linker.

Leong et al. (Protein Sci. 6(3): 609-17, Mar. 1997) disclose single-chain dimers of interleukin 8 linked via a peptide linker and with a single disulfide crosslink to prevent formation of multimers. The single-chain IL-8 dimers were designed to mimic the dimeric form of IL-8 in solution with the aim of producing heterodimer IL-8 variants.

Randal et al. (Protein Sci. 4:1057-60, Apr. 1998) disclose a single-chain variant of human interferon-gamma (IFN-γ) in which two IFN-γ chains are linked with an eight residue polypeptide linker. Landar et al. (J. Mol. Biol.. 299(1): 169-79, May 2000) disclose a biologically active single-chain mutant of IFN-γ produced by linking the two peptide chains of IFN-γ with a seven-residue linker and by mutating His111 in the first chain.

WO 98/27230 discloses various methods for polypeptide engineering using recursive sequence recombination (RSR), including single-chain versions of multisubunit factor proteins.

Multimeric polypeptides with polypeptide units joined by a cleavable linkage have also been disclosed for the purpose of producing monomeric polypeptides. For example, WO 00/17336 discloses a DNA cassette comprising two or more tandem repeating units of a nucleotide sequence encoding a biologically active short peptide attached to a linker peptide cleavable by a protease or a chemical agent. Expression of the DNA cassette results in a multimeric polypeptide that is subsequently cleaved enzymatically or chemically to result in the desired biologically active peptide.

U.S. Pat. No. 5,218,093 discloses biologically active variants of epidermal growth factor (EGF) comprising at least two monomeric EGF units that may be linked either by direct C-terminus to N-terminus fusion or through a cleavage-insensitive peptide linker. Human EGF is a monomeric protein having 53 amino acid residues, and the problem addressed by U.S. Pat. No. 5,218,093 is that of preventing undesired intracellular degradation by native proteases found in the recombinant microbial hosts used to produce recombinant EGF. According to this document, this avoids the need to produce recombinant EGF as a fusion protein bound to a stabilizing carrier, which requires subsequent downstream processing to release the EGF in monomeric form, or to produce EGF in multimeric form with repeating units linked through a cleavage-sensitive site that is subsequently digested using a site-specific enzyme. It is disclosed that the multimeric EGF has biological activity characteristic of monomeric EGF, but no other advantages aside from those specifically related to the production process are described.

U.S. Pat. No. 5,684,136 discloses a method for receptor activation by providing a conjugate comprising the direct fusion of a first ligand and a second ligand capable of binding to first and second receptors, and contacting the conjugate with the first and second receptors. The conjugate is in particular a chimeric molecule comprising a dimer of hepatocyte growth factor (HGF), a disulfide-linked heterodimer comprising a 69 kDa alpha subunit and a 34 kDa beta subunit.

WO 99/38891 discloses modified polypeptides with increased biological activity, in particular modified erythropoietin (EPO), in the form of multimeric polypeptides with polypeptide units covalently linked by thioester bonds.

WO 99/02710 discloses recombinant fusion protein multimers with altered biological activity such as increased plasma half-life, comprising two or more protein molecules fused directly or via a peptide linker, in particular modified EPO. This document teaches against conjugating proteins to chemical compounds or inert molecules as a way to increase biological activity, as this is said to often result in a significant decrease in the overall biological activity.

WO 92/06116 discloses a recombinant hematopoietic molecule comprising at least a portion of a first hematopoietic molecule having early myeloid differentiation activity and at least a portion of a second hematopoietic molecule having late myeloid differentiation activity, the recombinant hematopoietic molecule having early myeloid differentiation activity associated with the first hematopoietic molecule and late myeloid differentiation activity associated with the second hematopoietic molecule. The first hematopoietic molecule may be IL-3 or GM-CSF, and the second hematopoietic molecule may be EPO, G-CSF, IL-5 or M-CSF.

WO 01/03737 (published Jan. 18, 2001) discloses fusion proteins comprising a cytokine or growth factor fused to an immunoglobulin domain, in particular IgG. Also disclosed are multimeric fusion proteins comprising two or more members of the growth hormone superfamily joined with or without a peptide linker, although no such multimeric fusion proteins are actually exemplified.

Paige et al. (Pharm. Res. 12(12):1883-8, December 1995) disclose prolonged circulation of recombinant human granulocyte-colony stimulating factor (rhG-CSF) by covalently linking rhG-CSF to serum albumin through a heterobifunctional maleimido-carboxyl polyethylene glycol.

The present invention provides single-chain multimeric polypeptides with advantageous biological properties based on monomeric units of a polypeptide of a type that normally is biologically active in monomeric form, in particular based on polypeptide units that in their native form are non-glycosylated or have a relatively low degree of glycosylation.

### BRIEF DISCLOSURE OF THE INVENTION

In its broadest aspect, the present invention relates to single-chain multimeric polypeptides and polypeptide conjugates, in particular polypeptides and conjugates exhibiting agonist activity, comprising at least two monomeric units of a polypeptide of a type that is biologically active in monomeric form, as well as methods for preparation of the polypeptides and their use in medical treatment.

Accordingly, one aspect of the invention relates to single-chain multimeric polypeptide conjugate comprising at least two units of a monomeric polypeptide linked via a peptide bond or a peptide linker, wherein the monomeric polypeptide is of a type that is biologically active in monomeric form, and having at least one polymer moiety covalently bound to an attachment group of said polypeptide.

Another aspect of the invention relates to a single-chain multimeric polypeptide conjugate comprising at least two units of a monomeric polypeptide linked via a peptide bond or a peptide linker, wherein the monomeric polypeptide is of a type that is biologically active in monomeric form, and wherein at least one of said units differs from the corresponding wild-type polypeptide in that at least one amino acid residue comprising an attachment group for a non-polypeptide moiety has been introduced or removed, and having at least one non-polypeptide moiety covalently bound to an attachment group of said polypeptide.

A further aspect of the invention relate to nucleotide sequences encoding the single-chain polypeptides, expression vectors and host cells comprising such a nucleotide sequence and a method for preparing the polypeptides and conjugates by recombinant DNA techniques.

In still further aspects, the invention relates to a pharmaceutical composition comprising a polypeptide or conjugate of the invention together with a pharmaceutically acceptable excipient or vehicle, the use in therapy of a single-chain polypeptide or conjugate of the invention, and a method of treating a mammal using a single-chain polypeptide or conjugate of the invention.

### BRIEF DESCRIPTION OF THE DRAWINGS

FIG. 1 shows the in vivo biological activity of single-chain G-CSF dimers in healthy rats.

FIG. 2 shows the in vivo biological activity of single-chain G-CSF dimers in rats with chemotherapy-induced neutropenia.

### DETAILED DISCLOSURE OF THE INVENTION

Definitions

In the context of the present specification and claims, the following definitions apply:

The term “polypeptide” is understood to indicate a mature protein or a precursor form thereof as well as a functional fragment thereof which essentially has retained the activity of the mature protein, i.e. exhibits at least the same qualitative activity and preferably also at least a similar quantitative activity as the mature protein. A functional fragment may for instance be an N- and/or C-terminal truncated form of a full-length polypeptide, or an isoform, in particular a native isoform, of a full-length polypeptide.

The monomeric subunits of the polypeptides of the invention are derived from or otherwise made so as to mimic the structure and function of parent polypeptides which in their native form are biologically active as monomers, i.e. the parent polypeptides do not normally require formation of dimers, trimers, etc. in order to be biologically active in vivo.

The term “derived” is intended to indicate that the monomeric polypeptide subunit is prepared to mimic structural and/or functional properties of the corresponding native polypeptide in question. The “derived” polypeptide may have the same amino acid sequence as said native polypeptide, or it may have one or more amino acid changes, e.g. those made in accordance with the present invention. Thus, typically, the amino acid sequence of the “derived” polypeptide is at least 60% identical (homologous) to that of said native polypeptide, normally at least 70% identical, such as at least 80% or at least 90% or 95% identical. Preferably, the monomeric polypeptides used in the present invention are of mammalian origin, in particular of human origin. It will be understood that derived polypeptides include synthetic polypeptides with the necessary structural and/or functional similarity to a native polypeptide.

The terms “homology” and “identity” as used in connection with amino acid sequences are used in their conventional meanings. Amino acid sequence homology/identity is conveniently determined from aligned sequences (aligned by use of the CLUSTAL W Multiple Sequence Alignment Program, version 1.8, June 1999 (Thompson et al. (1994) CLUSTAL W: Improving the sensitivity of progressive multiple sequence alignment through sequence weighting, positions-specific gap penalties and weight matrix choice, Nucleic Acids Research, 22:4673-4680) using default parameters) or provided from the PFAM families database version 4.0 (http://pfam.wustl.edu/) (Nucleic Acids Res. Jan. 1, 1999; 27(1):260-2) by use of GENEDOC version 2.5 (Nicholas, K. B., Nicholas H. B. Jr., and Deerfield, D. W. II. 1997 GeneDoc: Analysis and Visualization of Genetic Variation, EMB-NEW.NEWS 4:14; Nicholas, K. B. and Nicholas H. B. Jr. 1997 GeneDoc: Analysis and Visualization of Genetic Variation).

The term “parent polypeptide” is used about the monomeric polypeptide, which in accordance with the present invention is provided in a modified single-chain form comprising two or more monomers. The individual monomers are linked by peptide bonds, optionally through a linker peptide, rather than being linked by non-covalent bonds or disulfide bonds. Accordingly, the single-chain polypeptides of the invention are expressed as a single polypeptide from a single nucleotide sequence rather than being expressed as single monomer molecules which are assembled to an oligomeric polypeptide only after expression. The parent monomer may be a wild-type monomeric polypeptide or a variant thereof, for instance a mutein form of the wild-type monomeric polypeptide which has been prepared by substitution or deletion of one or more amino acid residues thereof and/or insertion of one or more additional amino acid residues therein.

In the present context, the term “multimeric” or “oligomeric” polypeptide is merely intended to indicate that the polypeptide comprises two or more monomeric units that are linked together, (i.e. to form a dimer, trimer, tetramer., etc.) since, strictly speaking, the single-chain polypeptides of the invention cannot be said to be multimeric. The terms “dimer”, “dimeric”, “trimer”, “trimeric”, etc. are used in the same manner.

The term “signalling polypeptide” is used to denote a polypeptide that interacts with a cellular receptor so as to activate the receptor and thereby provide a signal initiating a signal transduction cascade in the cell carrying the receptor. Such a polypeptide is often also termed a ligand. In the present context, an “agonist” is a molecule which is capable of binding to a desired receptor to result in an activated receptor complex. An “antagonist” on the other hand is a molecule which is capable of binding to a desired receptor but incapable of mediating correct conformational changes of the receptor molecules necessary to result in an activated complex, whereby ligand-mediated receptor activation is substantially inhibited. Receptor activation upon binding of a suitable ligand generally involves conformational change in the receptor, e.g. oligomerisation of receptor subunits. A polypeptide of the invention “having agonist activity” refers to the fact that the multimeric polypeptides are able to bind to and activate at least one cellular receptor via at least one monomeric subunit, where said subunit typically is derived from or identical to a native agonist polypeptide having a similar agonist activity. Although the invention is primarily directed to polypeptides and conjugates that have agonist activity, it is also contemplated that the principles of the invention will be equally applicable to single-chain multimeric antagonist polypeptides, i.e. in which at least one monomeric unit of the polypeptide is able to bind to a target receptor without activating the receptor.

The term “ligand-binding domain” refers to the part or parts of a cellular receptor which is/are involved in specific recognition of and interaction with a receptor-binding site of an endogenous ligand. Analogously, the “receptor-binding site” is understood as those amino acid residues which are involved in polypeptide binding to the ligand-binding domain of the receptor. Normally, the receptor-binding site comprises 1-50 amino acid residues, such as 5-30 or 10-25 amino acid residues. The amino acid residues in question may be located in sequence, but are more often placed in spatial proximity to each other as a result of the folding of the polypeptide. In the Materials and Methods section below, a method is described for determining residues of a receptor-binding site.

The term “receptor” is understood to indicate a protein present on a cell surface which binds signalling molecules (i.e. ligands) as the first step in triggering the signal transduction cascade. Cell surface receptors are typically composed of different domains with different functions, such as an extracellular ligand-binding domain with which the signalling polypeptide interacts to initiate signal transduction, a transmembrane domain (or in some cases, several transmembrane domains) which anchors the receptor in the cell membrane, and an intracellular effector domain which generates a cellular signal in response to ligand binding (signal transduction).

The term “conjugate” (or interchangeably “conjugated polypeptide”) is intended to indicate a composite or chimeric molecule formed by the covalent attachment of one or more polypeptides to one or more non-peptide moieties. The term “covalent attachment” means that the polypeptide and the non-peptide moiety are either directly covalently joined to one another, or else are indirectly covalently joined to one another through an intervening moiety or moieties, such as a bridge, spacer, or linkage moiety or moieties. Preferably, the conjugated polypeptide is soluble at relevant concentrations and conditions, i.e. soluble in physiological fluids such as blood. Examples of conjugated polypeptides of the invention include glycosylated polypeptides and PEGylated polypeptides. The term “non-conjugated polypeptide” can be used about the polypeptide part of the conjugate.

The term “non-peptide moiety” is intended to indicate a molecule which is not a peptide and which is capable of conjugating to an attachment group of the polypeptide of the invention. Preferred examples of such molecule include polymers, e.g. polyalkylene oxide, oligosaccharide moieties, lipophilic groups, e.g. fatty acids, and ceramides. The “polymer molecule” is a molecule formed by covalent linkage of two or more monomers, wherein none of the monomers is an amino acid residue, except where the polymer is human albumin or another abundant plasma protein. The term “polymer” may be used interchangeably with the term “polymer molecule”. Except where the number of polymer molecules is expressly indicated, every reference to “a polymer”, “a polymer molecule”, “the polymer” or “the polymer molecule” contained in a polypeptide of the invention or otherwise used in the present invention shall be a reference to one or more polymer molecules.

The term “oligosaccharide moiety” is intended to indicate a carbohydrate-containing molecule comprising two or more monosaccharide residues, and which is capable of being attached to the polypeptide to produce a polypeptide conjugate in the form of a glycosylated polypeptide by way of in vivo or in vitro glycosylation. The term “in vivo glycosylation” is intended to mean any attachment of an oligosaccharide moiety occurring in vivo, i.e. during posttranslational processing in a glycosylating cell used for expression of the polypeptide, e.g. by way of N-linked and O-linked glycosylation. Usually, the N-glycosylated oligosaccharide moiety has a common basic core structure composed of five monosaccharide residues, namely two N-acetylglucosamine residues and three mannose residues. The exact oligosaccharide structure depends, to a large extent, on the glycosylating organism in question and on the specific polypeptide. Depending on the host cell in question the glycosylation may be classified as a high mannose type, a complex type or a hybrid type. The term “in vitro glycosylation” is intended to refer to a synthetic glycosylation performed in vitro, normally involving covalently linking an oligosaccharide moiety to an attachment group of a polypeptide, optionally using a cross-linking agent. In vivo and in vitro glycosylation are discussed in detail further below.

Although an oligosaccharide may normally be considered to be a polymer, in the context of the present specification and claims, “oligosaccharide moieties” are considered separately from “polymer molecules” as defined above, i.e. the term “polymer moiety” as used herein is not intended to encompass oligosaccharide moieties. It will be clear to persons skilled in the art in light of the present specification, however, that a polypeptide conjugate of the invention comprising one or more oligosaccharide moieties bound to an attachment group of a polypeptide, e.g. by way of in vivo glycosylation, may in addition comprise one or more polymer moieties bound to an attachment group of the polypeptide, e.g. by way of PEGylation.

In one embodiment, the multimeric polypeptide conjugate of the invention is one in which at least one monomeric polypeptide unit is non-glycosylated or is glycosylated at one, two or three amino acid residues, the glycosyl moieties typically having a molecular weight of about 1-10 kDa, more typically 1-6 kDa.. In many cases, this will apply to all of the monomeric units. Normally, the monomeric polypeptide units of the invention will be derived from monomeric polypeptides that in their native form are non-glycosylated or have a relatively low degree of glycosylation (a low degree of glycosylation being understood in the present context as glycosylation at not more than three sites on a monomeric polypeptide).

An “N-glycosylation site” has the sequence N-X′-S/T/C-X″, wherein X′ is any amino acid residue except proline, X″ is any amino acid residue that may or may not be identical to X′ and preferably is different from proline, N is asparagine and S/T/C is either serine, threonine or cysteine, preferably serine or threonine, and most preferably threonine. An “O-glycosylation site” is the OH-group of a serine or threonine residue.

The term “attachment group” is intended to indicate a functional group of the polypeptide, in particular of an amino acid residue thereof or an oligosaccharide moiety, capable of attaching a non-peptide moiety such as a polymer molecule, a lipophilic molecule or an organic derivatizing agent. Useful attachment groups and their matching non-peptide moieties are apparent from the table below.

| Attach- | | Examples | Conjugation | |
|---|---|---|---|---|
| ment | Amino | of non- | method/- | |
| group | acid | peptide moiety | Activated PEG | Reference |
| —NH₂ | N- | Polymer, e.g. | mPEG-SPA | Shearwater |
| | terminal, | PEG, with | Tresylated | Corp. Delgado |
| | Lys | amide or imine | mPEG | et al., critical |
| | | group | | reviews in |
| | | | | Therapeutic |
| | | | | Drug Carrier |
| | | | | Systems 9(3,4): |
| | | | | 249-304 |
| | | | | (1992) |
| —COOH | C- | Polymer, e.g. | mPEG-Hz | Shearwater |
| | terminal, | PEG, with ester | | Corp. |
| | Asp, Glu | or amide group | | |
| | | Oligo- | In vitro | |
| | | saccharide | coupling | |
| | | moiety | | |
| —SH | Cys | Polymer, e.g. | PEG- | Shearwater |
| | | PEG, with | vinylsulphone | Corp. Delgado |
| | | disulfide, | PEG- | et al., critical |
| | | maleimide or | maleimide | reviews in |
| | | vinyl sulfone | | Therapeutic |
| | | group | | Drug |
| | | Oligo- | In vitro | Carrier |
| | | saccharide | coupling | Systems 9(3,4): |
| | | moiety | | 249-304 |
| | | | | (1992) |
| —OH | Ser, Thr, | Oligo- | In vivo O- | |
| | OH—, | saccharide | linked | |
| | Lys | moiety | glycosylation | |
| | | PEG with ester, | | |
| | | ether, | | |
| | | carbamate, | | |
| | | carbonate | | |
| —CONH₂ | Asn as | Oligo- | In vivo N- | |
| | part of an | saccharide | glycosylation | |
| | N-glyco- | moiety | | |
| | sylation | Polymer, e.g. | | |
| | site | PEG | | |
| Aromatic | Phe, Tyr, | Oligo- | In vitro | |
| residue | Trp | saccharide | coupling | |
| | | moiety | | |
| —CONH₂ | Gln | Oligo- | In vitro | Yan and Wold, |
| | | saccharide | coupling | Biochemistry, |
| | | moiety | | 1984, Jul 31; |
| | | | | 23(16): |
| | | | | 3759-65 |
| Aldehyde | Oxidized | Polymer, e.g. | PEGylation | Andresz et al., |
| Ketone | oligo- | PEG, PEG- | | 1978, |
| | saccharide | hydrazide | | Makromol. |
| | | | | Chem. 179: |
| | | | | 301, |
| | | | | WO 92/16555, |
| | | | | WO 00/23114 |
| Guanidino | Arg | Oligo- | In vitro | Lundblad and |
| | | saccharide | coupling | Noyes, |
| | | moiety | | Chemical |
| | | | | Reagents for |
| | | | | Protein |
| | | | | Modification, |
| | | | | CRC Press |
| | | | | Inc., Florida, |
| | | | | USA |
| Imidazole | His | Oligo- | In vitro | As for |
| ring | | saccharide | coupling | guanidine |
| | | moiety | | |

For in vivo N-glycosylation, the term “attachment group” is used to indicate the amino acid residues that together constitute an N-glycosylation site. Although the asparagine residue of the N-glycosylation site is where the oligosaccharide moiety is attached during glycosylation, such attachment cannot be achieved unless the other amino acid residues of the N-glycosylation site are present. Accordingly, when the non-peptide moiety is an oligosaccharide moiety and the conjugation is to be achieved by N-glycosylation, the term “amino acid residue comprising an attachment group for the non-peptide moiety” as used in connection with alterations of the amino acid sequence of the polypeptide of interest is to be understood as meaning that one or more amino acid residues constituting an N-glycosylation site are to be altered in such a manner that either a functional N-glycosylation site is introduced into the amino acid sequence or removed from said sequence.

The term “comprising an attachment group” is intended to mean that the attachment group is present on an amino acid residue (including an N-glycosylation site) of the relevant peptide or polypeptide or on an oligosaccharide moiety attached to said peptide or polypeptide.

Amino acid names and atom names (e.g. CA, CB, NZ, N, O, C, etc.) are used herein as defined by the Protein DataBank (PDB) (www.pdb.org), which is based on the IUPAC nomenclature (IUPAC Nomenclature and Symbolism for Amino Acids and Peptides (residue names, atom names, etc.), Eur. J Biochem., 138, 9-37 (1984) together with their corrections in Eur. J. Biochem., 152, 1 (1985). The term “amino acid residue” is intended to indicate an amino acid residue contained in the group consisting of the 20 naturally occurring amino acids, i.e. alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W) and tyrosine (Tyr or Y). The terminology used for identifying amino acid positions/substitutions is illustrated as follows: T13 in a given amino acid sequence indicates position number 13 occupied by a threonine residue. T13K indicates that the threonine residue of position 13 has been substituted by a lysine residue. Multiple substitutions are indicated with a “+”, e.g. S93N+G95S/T means an amino acid sequence which comprises substitution of the serine residue in position 93 by an asparagine residue and substitution of the glycine residue in position 95 by a serine or a threonine residue.

The term “nucleotide sequence” is intended to indicate a consecutive stretch of two or more nucleotide molecules. The nucleotide sequence may be of genomic, cDNA, RNA, semi-synthetic or synthetic origin, or any combination thereof.

“Cell”, “host cell”, “cell line” and “cell culture” are used interchangeably herein and all such terms should be understood to include progeny resulting from growth or culturing of a cell. “Transformation” and “transfection” are used interchangeably to refer to the process of introducing DNA into a cell.

“Operably linked” refers to the covalent joining of two or more nucleotide sequences in such a manner that the normal function of the sequences can be performed. For example, the nucleotide sequence encoding a presequence or secretory leader is operably linked to a nucleotide sequence for a polypeptide if it is expressed as a preprotein that participates in the secretion of the polypeptide: a promoter or enhancer is operably linked to a coding sequence if it affects the transcription of the sequence.

The term “introduce” is primarily intended to mean substitution of an existing amino acid residue, but may also mean insertion of an additional amino acid residue. The term “remove” is primarily intended to mean substitution of the amino acid residue to be removed by another amino acid residue, but may also mean deletion (without substitution) of the amino acid residue to be removed.

The term “reduced immunogenicity” is intended to indicate that the conjugate gives rise to a measurably lower immune response than a reference molecule, e.g. a wild-type polypeptide, as determined under comparable conditions. The immune response may be a cell or antibody mediated response (see, e.g., Roitt: Essential Immunology (8th Edition, Blackwell) for further definition of immunogenicity). Normally, reduced antibody reactivity will be an indication of reduced immunogenicity. The reduced immunogenicity may be determined by use of any suitable method known in the art, e.g. in vivo or in vitro.

The term “functional in vivo half-life” is used in its normal meaning, i.e. the time at which 50% of the biological activity of the polypeptide or conjugate is still present in the body/target organ, or the time at which the activity of the polypeptide or conjugate is 50% of the initial value. As an alternative to determining functional in vivo half-life, “serum half-life” may be determined, i.e. the time in which 50% of the polypeptide or conjugate molecules circulate in the plasma or bloodstream prior to being cleared. Determination of serum half-life is often more simple than determining the functional in vivo half-life, and the magnitude of serum half-life is usually a good indication of the magnitude of functional in vivo half-life. Alternative terms to serum half-life include “plasma half-life”, “circulating half-life”, “serum clearance”, “plasma clearance” and “clearance half-life”. The polypeptide or conjugate is cleared by the action of one or more of the reticuloendothelial systems (RES), kidney, spleen or liver, by receptor-mediated degradation, or by specific or non-specific proteolysis. For injected protein pharmaceuticals, clearance is believed to be primarily determined by the level of renal clearance and receptor-mediated clearance, while e.g. non-specific proteolysis is believed to be of secondary importance. Normally, clearance depends on size (relative to the cutoff for glomerular filtration), charge, attached carbohydrate chains, the presence of cellular receptors for the protein and the affinity of the protein towards its receptor(s). The functional in vivo half-life and the serum half-life may be determined by any suitable method known in the art, e.g. by the method disclosed in U.S. Pat. No. 5,824,778.

The term “increased” as used about the functional in vivo half-life or serum half-life is used to indicate that the relevant half-life of the conjugate or polypeptide is statistically significantly increased relative to that of a reference molecule, e.g. a corresponding wild-type or non-conjugated monomeric polypeptide, as determined under comparable conditions. For instance, the relevant half-life may increased by at least 25%, such as by at least 50%, by at least 100%, or by at least 500% or 1000%.

The term “renal clearance” is used in its normal meaning to indicate any clearance taking place by the kidneys, e.g. by glomerular filtration, tubular excretion or tubular elimination. Renal clearance depends on physical characteristics of the conjugate, including molecular weight, size (diameter), symmetry, shape/rigidity and charge. Usually, a molecular weight of roughly about 66-67 kDa is considered to be a cut-off-value for renal clearance (although this can vary depending on e.g. the diameter and shape of the molecule, i.e. the “apparent size” as determined e.g. by SDS-PAGE). A reduced renal clearance may be established by any suitable assay, e.g. an established in vivo assay. Typically, the renal clearance is determined by administering a labelled (e.g. radiolabelled or fluorescence labelled) polypeptide conjugate to a patient and measuring the label activity in urine collected from the patient. The reduced renal clearance is determined relative to the corresponding non-conjugated polypeptide or the corresponding non-conjugated wild-type polypeptide under comparable conditions.

The term “function” is intended to indicate one or more specific functions of the polypeptide of interest and is generally to be understood qualitatively (i.e. having a similar function as the polypeptide of interest) and not necessarily quantitatively (i.e. the magnitude of the function is not necessarily similar). In the present context, the specific function of interest will in particular be one or more biological activities, e.g. in vitro or in vivo bioactivity.

The interchangeably used terms “measurable function” and “functional” are intended to indicate that the relevant function (preferably reflecting the intended use) of a conjugate of the invention may be detected when measured by standard methods known in the art, e.g. as in vitro and/or in vivo bioactivity. Typically, if not otherwise stated herein, a measurable function is at least 1%, such as at least 2% or 5%, preferably at least 10%, such as at least 25% or 50%, of that of the non-conjugated polypeptide as determined under comparable conditions, e.g. in the range of 1-1000%, such as 5-500% or 10-200% of the function of the non-conjugated polypeptide.

The interchangeably used terms “native” and “wild-type” are used about a polypeptide which has an amino acid sequence that is identical to one found in nature. The native polypeptide is typically isolated from a naturally occurring source, in particular a mammalian or microbial source, such as a human source, or is produced recombinantly by use of a nucleotide sequence encoding the naturally occurring amino acid sequence. The term “native” is intended to encompass allelic variants of the polypeptide in question. A “variant” is a polypeptide which has an amino acid sequence that differs from that of a native polypeptide in one or more amino acid residues. The variant is typically prepared by modification of a nucleotide sequence encoding the native polypeptide (e.g. to result in substitution, deletion or truncation of one or more amino acid residues of the polypeptide) or by introduction (by addition or insertion) of one or more amino acid residues into the polypeptide) so as to modify the amino acid sequence constituting said native polypeptide. A “fragment” is a part of a parent native or variant polypeptide, typically differing from such parent in one or more C-terminal or N-terminal amino acid residues or both types of such residues. Normally, the variant or fragment has retained at least one of the functions of the corresponding parent polypeptide, although as indicated above, the function of a variant or fragment need not be quantitatively comparable to that of the parent polypeptide.

Polypeptides and Conjugates of the Invention

In the context of the present description and claims, any reference to a polypeptide that is “biologically active in monomeric form” is intended to mean a cytokine, hormone, growth factor or other polypeptide with therapeutic activity that in its native form and in its native environment is mainly found in monomeric form in solution, and where the native monomeric polypeptide is biologically active in viva. This includes polypeptides where only one monomer is required for biological activity (typically polypeptides where binding of a single polypeptide elicits receptor activation) as well as polypeptides that occur as a monomer in vivo in physiological fluids, but where two, three or possibly more individual monomers together are responsible for receptor activation. An example of a polypeptide in this latter category is G-CSF, since native hG-CSF occurs in vivo as a monomer. Activation of the G-CSF receptor takes place via binding of two G-CSF molecules to a pair of G-CSF receptors, forming a 2:2 complex between the two receptor domains and the ligand, even though the two G-CSF molecules do not form a dimer as such (Aritomi et al., Nature, Vol. 401: 713-717, Oct. 14, 1999).

The monomeric polypeptides outlined above can be contrasted to polypeptides that normally occur in vivo in the form of e.g. dimers or trimers, for example with monomeric units bound together by disulfide bonds or hydrogen bonding, and where the formation of such multimers is a prerequisite for receptor binding or other biological activity. Polypeptides in this category can be either homomers, e.g. a homodimer comprising two identical monomers, or heteromers, e.g. a heterodimer comprising two different monomers.

One of the advantages of the single-chain polypeptides and conjugates of the invention based on polypeptides that are biologically active in monomeric form is that the single-chain multimeric polypeptides will normally have a reduced clearance and a longer circulation half-life than the corresponding monomeric polypeptides. This is due to the higher molecular weight of the multimeric polypeptide as well as possible attachment of non-polypeptide moieties.

The biological activity provided by the multimeric polypeptides and conjugates of the invention is in particular agonist activity, i.e. binding of a polypeptide of the invention to its receptor results in receptor activation, thereby providing a signal initiating a signal transduction cascade in the cell carrying the receptor.

Cytokines that in their native form have a monomeric structure in solution and are thus suitable for use in the single-chain polypeptides and conjugates of the invention include interleukins such as interleukin-1alpha (IL-1α), interleukin-1beta (IL-1β), interleukin-1ra (IL-1ra), interleukin-2 (IL-2), interleukin-3 (IL-3), interleukin-4 (IL-4), interleukin-6 (IL-6), interleukin-7 (IL-7), interleukin-8 (IL-8), interleukin-9 (IL-9), interleukin-11 (IL-11), interleukin-13 (IL-13), interleukin-15 (IL-15), interleukin 17 (IL-17) and interleukin 18 (IL-18); colony stimulating factors such as granulocyte colony stimulating factor (G-CSF) and granulocyte macrophage colony stimulating factor (GM-CSF); growth factors such as epidermal growth factor (EGF) and stem cell growth factor; interferons such as interferon alpha (INF-α) and interferon beta (INF-β); members of the tumour necrosis family such as tumour necrosis factor alpha (TNF-α), tumour necrosis factor beta (TNF-β) and osteoprotegerin ligand (OPGL); as well as e.g. erythropoietin (EPO) and human growth hormone.

Although the present invention is primarily directed to single-chain multimeric polypeptides based on polypeptides that are biologically active in monomeric form, it is also contemplated that the principles described herein will be applicable to polypeptides of the type that exist as dimers, trimers and other multimers in vivo. In this case, creating e.g. a single-chain dimeric polypeptide based on a polypeptide that normally exists and is active as a dimer in vivo might be of little significance in terms of the molecular weight of the polypeptide itself (with the possible exception of added weight provided by a relatively large linker sequence). However, creating single-chain versions of such polypeptides provides the possibility to individually modify the different monomers, e.g. by adding and/or removing attachment sites for non-polypeptide moieties. It is therefore contemplated that the principles of the present invention also can be applied to polypeptides which are biologically active in multimeric form to result in single-chain versions of such polypeptides wherein the individual monomers are linked by a peptide bond or a peptide linker, and in particular wherein one or more of the monomers have an amino acid sequence that is modified, in relation to the native sequence, by the introduction and/or removal of at least one attachment site for a non-polypeptide moiety. Examples of such polypeptides that are biologically active in multimeric form include IL-5 (homodimer), IL-10 (homodimer), IL-12 (heterodimer), IL-16 (homodimer), interferon gamma (dimer), vascular endothelial growth factor (VEGF; homodimer), and human fertility hormones such as follicle stimulating hormone (FSH; heterodimer). As explained below, it has been found that single-chain polypeptides having a reduced in vitro bioactivity (reduced receptor binding affinity) also show an increased in vivo half-life that can be attributed to a lower rate of receptor-mediated clearance (RMC). In a particular embodiment, the invention therefore relates to a general method for reducing receptor-mediated clearance of a polypeptide, compared to the relevant wild-type polypeptide, by producing the polypeptide in the form of a single-chain construct comprising two or more monomeric units linked by a peptide bond or peptide linker. This general method is contemplated to be applicable to polypeptides that in their native form are biologically active as monomers as well as to polypeptides that are biologically active as multimers.

While the polypeptides used according to the invention may be of any origin, they are normally of mammalian origin, in particular of human origin.

The multimeric polypeptides and conjugates of the invention comprise two or more monomeric polypeptide units with similar biological activity, in particular two or more monomeric polypeptide units that have substantially the same biological activity and which may be substantially similar in sequence. The term “similar biological activity” refers to the fact that the two or more monomeric units should be of the same type of molecule and have the same type of biological activity or at least be derived from a molecule with the same type of biological activity. Although the individual monomeric units may in some cases have an identical amino acid sequence, it will be clear that the individual sequences need not be identical and that the level of their biological activity need not be identical. To illustrate using G-CSF (granulocyte colony stimulating factor) as an example, a dimeric polypeptide according to the invention may comprise, e.g., two identical monomers of wild-type G-CSF, a wild-type G-CSF monomer and a variant in which one or more amino acids have been inserted and/or removed relative to the wild-type G-CSF monomer, two G-CSF variants (which may be identical or different from each other), a wild-type G-CSF monomer and a fragment thereof, a G-CSF variant and a fragment of the variant or of the wild-type monomer, etc. The same principles for combining different monomeric units are of course also valid for other multimers such as trimers, tetramers, pentamers, etc. In the case of a trimer, for example, it may comprise three identical monomeric units, two identical monomeric units (wild-type or variant) and one unit which is different from the two (e.g. a variant or fragment), or three different units. When the individual monomers are identical the polypeptide is termed a “homomer”, and when they are different from each other the polypeptide is termed a “heteromer”.

The monomers used for constructing the multimeric polypeptide may be linked by a peptide bond, or may be connected by a suitable linker peptide. If used, the linker peptide should be of a type (length, amino acid composition, amino acid sequence, etc.) that is adequate to link two (or more) monomers in such a way that they assume a conformation relative to one another so that the resulting multimeric polypeptide has the desired activity. Furthermore, the linker peptide is typically designed to increase the stability of the resulting multimeric polypeptide towards proteolytic degradation, e.g. by use of special amino acid sequences or residues not readily subject to proteolysis. When the multimeric polypeptide is intended for conjugation to a non-polypeptide moiety, the peptide linker sequence may comprise one or more attachment groups for said non-polypeptide moiety. For instance, when the non-polypeptide moiety is an oligosaccharide moiety the linker can contain a sequence that provides an N-glycosylation site. When the non-polypeptide moiety is PEG, the linker can contain e.g. Lys or Cys.

The linker peptide will often predominantly include the amino acid residues Gly, Ser, Ala and/or Thr. The linker typically comprises 1-30 amino acid residues, such as a sequence of about 2-20 or 3-15 amino acid residues. Likewise, the amino acid residues selected for inclusion in the linker peptide should exhibit properties that do not interfere significantly with the activity of the multimeric polypeptide. Thus, the linker peptide should on the whole not exhibit a charge which would be inconsistent with the desired activity of the multimeric polypeptide, or interfere with internal folding, or form bonds or other interactions with amino acid residues in one or more of the monomers which would seriously impede the binding of the multimeric polypeptide to the ligand-binding domain of the receptor.

Specific linkers for use in the present invention may be designed on the basis of known naturally occurring as well as artificial polypeptide linkers (see, e.g., Hallewell et al. (1989), J. Biol. Chem. 264, 5260-5268; Alfthan et al. (1995), Protein Eng. 8, 725-731; Robinson & Sauer (1996), Biochemistry 35, 109-116; Khandekar et al. (1997), J. Biol. Chem. 272, 32190-32197; Fares et al. (1998), Endocrinology 139, 2459-2464; Smallshaw et al. (1999), Protein Eng. 12, 623-630; U.S. Pat. No. 5,856,456). For instance, linkers used for creating single-chain antibodies, e.g. a 15mer consisting of three repeats of a Gly-Gly-Gly-Gly-Ser amino acid sequence ((Gly₄Ser)₃), are contemplated to be useful in the present invention. Other linkers that contemplated to be useful in the present invention are GlySerThrSerGly-SerSerGlyLysSerSerGluGlyLysGly, and GlyGlyGlyGlySerGlyGlyGlyAsnSerThrGlyGly-GlySer, the latter being an example of a linker providing a glycosylation site (AsnSerThr). Furthermore, phage display technology as well as selective infective phage technology can be used to diversify and select appropriate linker sequences (Tang et al., J. Biol. Chem. 271, 15682-15686, 1996; Hennecke et al. (1998), Protein Eng. 11, 405-410). Also, Arc repressor phage display has been used to optimize the linker length and composition for increased stability of the single-chain protein (Robinson and Sauer (1998), Proc. Natl. Acad. Sci. USA 95, 5929 5934).

Another way of obtaining a suitable linker is by optimizing a simple linker, e.g. ((Gly₄Ser)ₙ), through random mutagenesis. It will be clear from the present specification that whatever the nature of the linker, it should be one which is not readily susceptible to cleavage by e.g. proteases or chemical agents, since cleavage of the multimeric polypeptide to result in two or more monomeric units is not desired in the present context.

As indicated above, in one embodiment the multimeric polypeptide conjugate comprises two or more monomeric polypeptide units with the same amino acid sequence, and with at least one non-polypeptide moiety attached to an attachment group thereof. In this case, the multimeric polypeptide may comprise two or more wild-type monomeric units linked together so as to obtain a multimeric polypeptide having a sufficiently high molecular weight, i.e. without any monomeric units that differ from the wild-type polypeptide, other than the presence of one or more attached non-polypeptide moieties. Alternatively, the polypeptide may comprise two or more units that are modified, in relation to the relevant wild-type amino acid sequence, by insertion and/or removal of one or more amino acid residues, e.g. by substitution of one or more amino acid residues in the native sequence with a non-native amino acid residue.

In another embodiment, the multimeric polypeptide comprises two or more monomeric polypeptide units with different amino acid sequences. As indicated above, this can e.g. for the case of a dimer be a wild-type unit and a modified unit or two modified units that are different from each other. A single-chain multimeric polypeptide with non-identical monomeric units provides the advantage that the individual units can be designed to give different desired characteristics to the multimeric polypeptide. For example, the amino acid sequence of one of the monomeric units of a dimeric polypeptide of the invention can be designed for optimal binding to a receptor, while the amino acid sequence of the other monomeric unit can be designed to provide optimal attachment sites for non-polypeptide moieties in order to obtain desired properties of molecular weight, bulk, etc. and thus desired properties in terms of e.g. half-life.

It will be clear that at least one of the monomeric units of the multimeric polypeptide must be biologically active, i.e. capable of binding to the intended target receptor with a sufficient binding affinity to elicit a desired receptor activation. In some cases it may be sufficient that only one of the monomeric units is biologically active, so that the other monomeric unit(s) has/have a significantly reduced agonist activity or binding affinity or perhaps substantially no activity or binding affinity, although it is generally preferred that both (or all) of the monomeric units are biologically active and are able to bind to and activate the intended receptor. For a multimeric polypeptide of the invention having at least first and second monomeric subunits, wherein the first monomeric subunit is biologically active as defined above, this means that the second monomeric subunit typically has an amino acid sequence homology to the first monomeric subunit of at least about 70%, preferably at least about 80%, more preferably at least about 85%, still more preferably at least about 90%, such as at least about 95%. The same applies to any additional monomeric unit in the case of a trimer or higher multimer. The degree of homology may conveniently be determined using the CLUSTAL W sequence alignment program referred to above.

Further, it will be clear that the individual monomeric subunits of the single-chain multimeric polypeptide will, in the case of a fragment of a full-length polypeptide, have a certain minimum length in relation to the relevant wild-type polypeptide. Thus, each monomeric subunit will typically have a length, determined as the number of amino acid residues compared to the number of amino acid residues in the corresponding wild-type polypeptide (e.g. the relevant wild-type human polypeptide) of at least about 50%, preferably at least about 60%, more preferably at least about 70%, still more preferably at least about 80%, such as at least about 90% or 95% of the length of the wild-type polypeptide.

By removing and/or introducing one or more amino acid residues comprising an attachment group for a non-polypeptide moiety, it is possible to specifically adapt the polypeptide so as to make the molecule more susceptible to conjugation to the non-polypeptide moiety of choice, to optimize the conjugation pattern (e.g. to ensure an optimal distribution of non-polypeptide moieties on the surface of the molecule and to ensure that only the attachment groups intended to be conjugated are present in the molecule) and thereby obtain a conjugate molecule with improved activity and/or other properties, e.g. reduced immuno-genicity.

In embodiments with one or more modified monomeric units, the amino acid sequence of the wild-type polypeptide and the amino acid sequence of the monomeric unit of the invention may differ in that at least one and preferably more, e.g. 2-15, amino acid residues comprising an attachment group for a non-polypeptide moiety have been introduced into the monomeric unit, preferably by substitution, compared to the wild-type amino acid sequence. Thereby, for instance, shielding by non-polypeptide moieties may be achieved in more or different regions of the polypeptide molecule, leading to a lower immune response, and/or the molecular weight, shape, size and/or charge of the conjugate may be optimised. Preferably, such amino acid residue is introduced in an amino acid located at the surface of the polypeptide, more preferably in a position occupied by an amino acid residue having more than 25%, such as more than 50% or even more than 75% of its side chain exposed at the surface of the molecule. As used herein, the term “surface-exposed” refers to an amino acid residue with a side chain that is at least partially exposed at the surface of the molecule, in particular at least 25% exposed.

A method for determining the percentage exposed surface area of side chains of amino acid residues, and thus for identifying suitable positions for modification, based on an analysis of the 3D structure of the polypeptide, is given in the Materials and Methods section below. Alternatively or additionally, the position to be modified may be identified on the basis of an analysis of the sequence family of the polypeptide in question. More specifically, the position to be modified can be one that in one or more members of the family other than the parent polypeptide is occupied by an amino acid residue comprising the relevant attachment group (when such amino acid residue is to be introduced) or which in the parent polypeptide, but not in one or more other members of the family, is occupied by an amino acid residue comprising the relevant attachment group (when such amino acid residue is to be removed).

In order to determine an optimal distribution of attachment groups, the distance between amino acid residues located at the surface of the polypeptide is calculated on the basis of the polypeptide's 3D structure. More specifically, the distance between the CB's of the amino acid residues comprising such attachment groups, or the distance between the functional group (NZ for lysine, CG for aspartic acid, CD for glutamic acid, SG for cysteine) of one and the CB of another amino acid residue comprising an attachment group are determined. In case of glycine, CA is used instead of CB. In polypeptides according to the invention, any of said distances is preferably more than 8 521 , in particular more than 10 Å, in order to avoid or reduce heterogeneous conjugation.

In another embodiment, one difference between the amino acid sequence of the wild-type polypeptide and the amino acid sequence of the monomeric unit used herein is that one or preferably more, e.g. 2-15, amino acid residues comprising an attachment group for a non-polypeptide moiety have been removed, preferably by substitution, from the wild-type amino acid sequence. The amino acid residue to be removed is preferably one to which conjugation is disadvantageous, e.g. an amino acid residue located at or near a functional site of the polypeptide (since conjugation at such a site may result in inactivation or reduced activity of the resulting conjugate due to impaired receptor recognition). In the present context the term “functional site” is intended to indicate one or more amino acid residues which is/are essential for or otherwise involved in the function or performance of the polypeptide, in particular receptor binding and/or activation. The functional site may be determined by methods known in the art and is preferably identified by analysis of a structure of the polypeptide complexed to a relevant receptor.

In preferred embodiments of the present invention, more than one amino acid residue of at least one monomeric unit is altered, e.g. the alteration embraces removal as well as introduction of amino acid residues comprising an attachment group for the non-polypeptide moiety of choice. In a particular embodiment, the amino acid sequence of the monomeric polypeptide unit may differ from the relevant wild-type amino acid sequence in that a) at least one amino acid residue comprising an attachment group for the non-polypeptide moiety and present in the wild-type amino acid sequence has been removed, preferably by substitution, and b) at least one amino acid residue comprising an attachment group for the non-polypeptide moiety has been introduced into the amino acid sequence, preferably by substitution. This embodiment is considered of particular interest in that it is possible to specifically design the polypeptide so as to obtain an optimal conjugation to the non-polypeptide moiety of choice. For instance, by introducing and removing selected amino acid residues, e.g. as exemplified below for G-CSF, it is possible to ensure an optimal distribution of attachment groups for the non-polypeptide moiety of choice, which gives rise to a conjugate in which the non-polypeptide moieties are placed so as to effectively shield epitopes and other surface parts of the polypeptide without substantially impairing the function of the polypeptide.

In one alternative of this embodiment, it is possible to produce a multimeric polypeptide according to the invention in which, taking a dimer as an example, one of the monomeric units comprises either a wild-type sequence or a variant sequence modified such that one or more attachment sites for a non-polypeptide moiety have been removed as compared to the wild-type, thereby avoiding attachment of undesired non-polypeptide moieties to this monomeric unit (although it may, if desired, maintain any naturally occurring glycosylation sites so as to allow glycosylation corresponding to the native polypeptide), while the other monomeric unit comprises a sequence with one or more attached non-polypeptide moieties. In this case, the latter monomeric unit may have an amino acid sequence corresponding to that of the wild-type, but with one or more polymer moieties attached thereto, e.g. PEG moieties, or it may be a variant with introduced and/or removed attachment groups for polymer and/or oligosaccharide moieties. The advantage of this approach is that the resulting multimeric polypeptide comprises a monomeric unit corresponding in sequence and possible glycosylation to the native polypeptide together with a monomeric unit modified with one or more attachment groups, and possibly with additional amino acid modifications, so as to give the overall monomeric polypeptide the desired properties in terms of biological activity, molecular weight, half-life, epitope shielding, etc.

In addition to the removal and/or introduction of amino acid residues, the polypeptide may comprise other substitutions or glycosylations which are not related to introduction and/or removal of amino acid residues comprising an attachment group for the non-polypeptide moiety.

In order to avoid too much disruption of the structure and function of the parent molecule, the total number of amino acid residues to be altered in accordance with the present invention, e.g. as exemplified for G-CSF below, will typically not exceed 15. The exact number of amino acid residues and the type of amino acid residues to be introduced and/or removed depend, i.a., on the desired nature and degree of conjugation (e.g. the identity of the non-polypeptide moiety, how many non-polypeptide moieties it is desirable or possible to conjugate to the polypeptide, where in the polypeptide conjugation should be performed or avoided, etc.). Preferably, the polypeptide part of the conjugate of the invention or the polypeptide of the invention comprises an amino acid sequence which differs in 1-15 amino acid residues from the native amino acid sequence such as in 1-8 or 2-8 amino acid residues, e.g. in 1-5 or 2-5 amino acid residues. Thus, normally the polypeptide part of the conjugate or the polypeptide of the invention comprises an amino acid sequence which differs from the native amino acid sequence in 1, 2, 3, 4, 5, 6, 7, 8, 9, 10, 11, 12, 13, 14 or 15 amino acid residues.

The amino acid residue comprising an attachment group for a non-polypeptide moiety, whether it is removed or introduced, is selected on the basis of the nature of the non-polypeptide moiety of choice and, in most instances, on the basis of the method in which conjugation between the polypeptide and the non-polypeptide moiety is to be achieved. It will be understood that in order to preserve a measurable function of the conjugate or polypeptide, amino acid residues to be modified (by deletion, preferably by substitution) are selected from those amino acid residues which are not essential for providing a measurable activity. Accordingly, amino acid residues to be modified are different from those required for receptor binding or activation.

Amino acid residue modifications in one or more monomeric units of the multimeric polypeptides of the invention are preferably selected from among the following:

introduction of a lysine residue, typically substitution in place of a non-lysine residue,

removal of a lysine residue, in particular introduction of an arginine or glutamine residue in place of a lysine residue,

introduction of a cysteine residue, typically substitution in place of a non-cysteine residue,

introduction of an aspartic acid residue, typically substitution in place of a non-aspartic acid residue,

introduction of a glutamic acid residue, typically substitution in place of a non-glutamic acid residue,

removal of a cysteine, aspartic acid or glutamic acid residue, typically substitution by another amino acid residue,

introduction and/or removal of a histidine residue, typically by substitution,

introduction of an N- or O-glycosylation site, and

removal of an N- or O-glycosylation site.

The conjugate of the present invention will normally have one or more improved properties as compared to the native polypeptide, including increased functional in vivo half-life, increased serum half-life, reduced clearance, reduced immunogenicity and/or increased bioavailability. Consequently, medical treatment with a conjugate of the invention offers a number of advantages over the currently available compounds, including longer duration between injections and fewer side effects.

As explained above, the increased functional in vivo half-life is normally obtained as a consequence of the conjugate of the invention having a reduced susceptibility to renal and/or receptor-mediated clearance as compared to the native monomeric polypeptide, in part as a result of an increased molecular weight. It should be noted in this regard that the actual molecular weight does not necessarily have to be above any certain limit, e.g. 60 or 65 kDa, in order to achieve reduced renal clearance, since renal clearance depends not only on the molecular weight as such, but also on e.g. on the three-dimensional structure and bulk of the conjugate. Thus, for purposes of reduced renal clearance, it is sufficient that the “apparent size” of the conjugate, e.g. as determined by SDS-PAGE, is sufficiently high, e.g. at least about 60 or 65 kDa, such as at least about 66 or 67 kDa. Polymer molecules such as PEG have been found to be particularly useful for adjusting the molecular weight of the conjugate and for providing a sufficiently high apparent size. In conjugates of the invention, each monomeric polypeptide unit will often have a molecular weight (exclusive of any polymer moiety bound to said unit) of less than about 34 kDa, such as less than about 30 kDa.

In one embodiment, the multimeric polypeptide conjugate of the invention thus comprises at least one non-polypeptide moiety bound to an attachment group of at least one of the monomeric polypeptide units, such that the apparent size of the multimeric polypeptide conjugate is at least about 60 kDa, such as at least about 65 kDa, e.g. at least about 67 kDa.

Generally, activation of the receptor is associated with receptor-mediated clearance (RMC) such that binding of a polypeptide to its receptor without activation does not lead to RMC, while activation of the receptor leads to RMC. The clearance is due to internalisation of the receptor-bound polypeptide with subsequent lysosomal degradation. Reduced RMC may be achieved by designing the conjugate so as to be able to bind and activate a sufficient number of receptors to obtain optimal in vivo biological response and avoid activation of more receptors than required for obtaining such response. This may be reflected in reduced in vitro bioactivity and/or increased off-rate.

Typically, reduced in vitro bioactivity reflects reduced efficacy/efficiency and/or reduced potency and may be determined by any suitable method for determining any of these properties. For instance, in vitro bioactivity may be determined in a luciferase based assay (see Materials and Methods).

For G-CSF it has been found that a relatively low in vitro bioactivity, compared to the bioactivity of hG-CSF (SEQ ID NO:1), is advantageous in terms of both a long plasma half-life and a high degree of stimulation of neutrophils. This is explained in further detail below.

Single-chain G-CSF Polypeptides and Conjugates

A preferred single-chain polypeptide conjugate of the invention is one comprising two or more subunits of a G-CSF polypeptide, e.g. two, three or four subunits, although normally the multimeric polypeptide will contain two subunits. In the case of two subunits, these may be two monomers each comprising the amino acid sequence of hG-CSF as shown in SEQ ID NO:1, a monomer having the sequence of hG-CSF together with a monomer having an amino acid sequence that is altered relative to hG-CSR, or two monomers with altered amino acid sequences relative to hG-CSF.

One embodiment of this aspect of the invention thus relates to a single-chain multimeric polypeptide having G-CSF activity, comprising at least two monomeric units independently selected from (a) hG-CSF with the amino acid sequence shown in SEQ ID NO:1 and (b) variants of hG-CSF, said monomeric units being linked via a peptide bond or a peptide linker, wherein the polypeptide has at least one non-polypeptide moiety covalently bound to an attachment group of the polypeptide.

Another embodiment of this aspect of the invention relates to a single-chain multimeric polypeptide having G-CSF activity, comprising at least two monomeric units independently selected from (a) hG-CSF with the amino acid sequence shown in SEQ ID NO:1 and (b) variants of hG-CSF, said monomeric units being linked via a peptide bond or a peptide linker, wherein the polypeptide has at least one non-polypeptide moiety covalently bound to an attachment group of the polypeptide and exhibits an in vitro bioactivity in the range of about 2-30% of the bioactivity of non-conjugated hG-CSF as determined by the luciferase assay described herein.

In a further embodiment, the invention relates to a single-chain multimeric polypeptide having G-CSF activity, comprising at least two monomeric units independently selected from (a) hG-CSF with the amino acid sequence shown in SEQ ID NO:1 and (b) variants of hG-CSF, said monomeric units being linked via a peptide bond or a peptide linker, the polypeptide comprising at least one covalently bound polymer molecule selected from the group consisting of linear and branched polyalkylene oxides.

In a particular embodiment, the invention relates to a single-chain multimeric polypeptide having G-CSF activity, comprising at least two monomeric units independently selected from (a) hG-CSF with the amino acid sequence shown in SEQ ID NO:1 and (b) variants of hG-CSF, said monomeric units being linked via a peptide bond or a peptide linker, the polypeptide comprising at least one covalently bound polyethylene glycol molecule.

Unless otherwise indicated, the term “G-CSF” as used herein is intended to refer to polypeptides comprising the amino acid sequence of wild-type human G-CSF as set forth in SEQ ID NO:1 as well as variants thereof with one or more changes in the form of substitutions, additions/insertions or deletions compared to SEQ ID NO:1. Such G-CSF variants are disclosed in detail below.

The amino acid residue modifications in G-CSF variant monomeric units may in particular be selected from the group consisting of introduction of a lysine, cysteine, aspartic acid, glutamic acid or histidine residue, and removal of a lysine, cysteine, aspartic acid, glutamic acid or histidine residue. Alternatively or additionally, at least one of the monomers of the single-chain multimeric G-CSF polypeptide of this aspect of the invention may comprise at least one amino acid residue modification selected from the group consisting of introduction of an N- or O-glycosylation site, and removal of an O-glycosylation site.

In another embodiment, the single-chain multimeric G-CSF polypeptide comprises at least two G-CSF polypeptide monomers linked via a peptide bond or a peptide linker, wherein at least one of said monomers is a variant of wild-type human G-CSF comprising at least one introduced attachment site for a polymer moiety.

In a further embodiment, the single-chain multimeric G-CSF polypeptide comprises at least two G-CSF polypeptide monomers linked via a peptide bond or a peptide linker, wherein at least one of said monomers is a variant of wild-type human G-CSF wherein at least one attachment site for a non-polypeptide moiety has been introduced in a position that in wild-type human G-CSF is occupied by a surface-exposed amino acid residue.

In a still further embodiment, the single-chain multimeric G-CSF polypeptide comprises at least two G-CSF polypeptide monomers linked via a peptide linker, wherein the peptide linker comprises at least one amino acid residue comprising an attachment group for a non-polypeptide moiety.

As described in the literature, the principal biological effect of G-CSF in vivo is to stimulate the growth and development of neutrophils (Welte et al., PNAS USA 82:1526-1530, 1985, Souza et al., Science, 232:61-65, 1986). The amino acid sequence of human G-CSF (hG-CSF) was reported by Nagata et al., Nature 319:415-418, 1986, and is shown in the appended SEQ ID NO:1.

Recombinant human G-CSF (rhG-CSF) is generally used for treating various forms of leukopenia/neutropenia, i.e. a reduced white blood cell count that arises e.g. as a result of chemotherapy. Since leukopenia is a serious side effect of chemotherapy that increases the risk of infection and can reduce the effectiveness of chemotherapy, it is important to be able to reduce the time period during which patients are subject to leukopenia.

Commercial preparations of rhG-CSF are available under the names filgrastim (Gran® and Neupogen®), lenograstim (Neutrogin® and Granocyte®) and nartograstim (Neu-up®). Gran® and Neupogen® are non-glycosylated and produced in recombinant E. coli cells. Neutrogin® and Granocyte® are glycosylated and produced in recombinant CHO cells and Neu-up® is non-glycosylated with five amino acids substituted at the N-terminal region of intact rhG-CSF produced in recombinant E. coli cells.

The commercially available rhG-CSF has a short-term pharmacological effect and must often be administered more than once per day for the duration of the leukopenic state. A molecule with a longer circulation half-life would decrease the number of administrations necessary to alleviate the leukopenia and prevent consequent infections. Given the potential for obtaining more optimal therapeutic hG-CSF levels with concomitant enhanced therapeutic effect using less fewer injections, there is clearly a need for improved hG-CSF-like molecules.

As indicated above, it has been found that a relatively low in vitro G-CSF bioactivity is advantageous. In a preferred embodiment, the in vitro bioactivity of a multimeric G-CSF conjugate of the invention is in the range of about 1-30%, preferably about 2-30%, of the bioactivity of non-conjugated hG-CSF as determined by the luciferase assay described herein. The in vitro bioactivity of such conjugates is thus preferably reduced by at least about 70%, such as by at least about 75%, e.g. by at least about 80% or 85%, as compared to the in vitro bioactivity of hG-CSF, determined under comparable conditions. Expressed differently, the conjugate may have an in vitro bioactivity that is as small as about 1%, typically at least about 2%, such as at least about 3%, 4% or 5%, of that of the corresponding non-conjugated hG-CSF polypeptide. For instance, the in vitro bioactivity may be in the range of about 2-30% of that of the reference polypeptide, e.g. about 3-25% or 4-20%, determined under comparable conditions. In cases where reduced in vitro bioactivity is desired in order to reduce receptor-mediated clearance, it will be clear that sufficient bioactivity to obtain the desired receptor activation must be nevertheless be maintained, which is why the bioactivity should be at least about 1-2% of that of hG-CSF and preferably slightly higher as given above.

In a preferred embodiment, a single-chain multimeric G-CSF conjugate is in the form of a dimer with either two wild-type monomeric units or with one or possibly two units which are variants of the wild-type human G-CSF (hG-CSF), and preferably attached to one or more PEG moieties.

In addition to the above considerations regarding in vitro bioactivity, it has further been found that advantageous results are obtained when the apparent size (also referred to as the “apparent molecular weight” or “apparent mass”) of the polypeptide conjugates of the invention, or at least a majority of such conjugates, is at least about 50 kDa, preferably at least about 55 kDa, more preferably at least about 60 kDa, e.g. at least about 66 kDa. This is believed to be due to the fact that renal clearance is substantially eliminated for conjugates having a sufficiently large apparent size. In the present context, the “apparent size” of a G-CSF conjugate or polypeptide is determined by SDS-PAGE as described in the examples section below.

It will be understood that the apparent size in kDa of a conjugate or polypeptide is not necessarily the same as the actual molecular weight of the conjugate or polypeptide. Rather, the apparent size is a reflection of both the actual molecular weight and the overall bulk. Since, in most cases, attachment of one or more PEG groups or other non-polypeptide moieties will result in a relatively large increase of the bulk of the polypeptide to which such moieties are attached, the polypeptide conjugates of the invention will normally have an apparent size that exceeds the actual molecular weight of the conjugate. Therefore, in connection with renal clearance, a conjugate of the invention can easily exhibit properties characteristic of a polypeptide with a molecular weight above e.g. 50 kDa (corresponding to the apparent size) but have an actual molecular weight below 50 kDa.

In a further preferred embodiment, the multimeric G-CSF conjugates of the invention have both an apparent size of at least about 50 kDa and a reduced in vitro bioactivity (reduced receptor binding affinity) compared to hG-CSF as explained above. It has been found that such conjugates have both a low renal clearance as a result of the large apparent size and a low receptor-mediated clearance as a result of the low in vitro bioactivity (low receptor binding affinity). The overall result is excellent performance in terms of effective stimulation of neutrophils together with a significantly increased in vivo half-life and thus a long duration of action that provides important clinical advantages.

In the following, the invention will be illustrated by way of example with reference to different variants based on hG-CSF. For the sake of simplicity, the numbering of the possible amino acid modifications below will be with reference to the known amino acid sequence of the hG-CSF monomer as shown in SEQ ID NO:1. It will be understood that the modifications illustrated below may, according to the properties desired in any given case, be performed in either the C-terminal or N-terminal monomeric unit or both. Further details regarding G-CSF variants and methods for producing such variants are found in PCT/DK01/00011 and U.S. Ser. No. 09/760,008, which are hereby incorporated by reference.

Conjugate of the Invention Wherein the Non-polypeptide Moiety is Attached to a Lysine or the N-terminal Amino Acid Residue

In a preferred embodiment the conjugate of the invention is one wherein the amino acid residue comprising an attachment group for the non-polypeptide moiety is a lysine residue and the non-polypeptide moiety is any molecule which has lysine as an attachment group. For instance, the non-polypeptide moiety may be a polymer molecule, in particular any of the molecules mentioned in the section entitled “Conjugation to a polymer molecule”, and preferably selected from the group consisting of linear or branched polyethylene glycol or another polyalkylene oxide. Most preferably, the polymer molecule is a PEG such as mPEG-SPA (Shearwater Corp.) or oxycarbonyl-oxy-N-dicarboxyimide PEG (U.S. Pat. No. 5,122,614).

i) Introduction of Lysine Residues

In order to obtain a more extensive or differently distributed conjugation, it may be desirable to introduce at least one non-naturally occurring lysine residue, in particular in a position which is occupied by an amino acid residue having a side chain which is more than 25% surface exposed and not part of a cystine or located at a receptor binding site.

Accordingly, in one embodiment the conjugate of the invention is one which comprises a non-polypeptide moiety having lysine as attachment group and a polypeptide comprising an amino acid sequence that differs from the native sequence in that at least one lysine residue has been introduced. Residues are in particular introduced such that they have more than 50% of the side chain surface exposed.

In a dimeric G-CSF conjugate, one or both of the monomeric units may thus have an amino acid sequence that differs from the amino acid sequence shown in SEQ ID NO:1 in at least one substitution selected from the group consisting of T1K, P2K, L3K, G4K, P5K, A6K, S7K, S8K, L9K, P10K, Q11K, S12K, F13K, L14K, L15K, E19K, Q20K, V21K, R22K, Q25K, G26K, D27K, A29K, A30K, E33K, A37K, T38K, Y39K, L41K, H43K, P44K, E45K, E46K, V48K, L49K, L50K, H52K, S53K, L54K, I56K, P57K, P60K, L61K, S62K, S63K, P65K, S66K, Q67K, A68K, L69K, Q70K, L71K, A72K, G73K, S76K, Q77K, L78K, S80K, F83K, Q86K, G87K, Q90K, E93K, G94K, S96K, P97K, E98K, L99K, G100K, P101K, T102K, D104K, T105K, Q107K, L108K, D109K, A111K, D112K, F113K, T115K, T116K, W118K, Q119K, Q120K, M121K, E122K, E123K, L124K, M126K, A127K, P128K, A129K, L130K, Q131K, P132K, T133K, Q134K, G135K, A136K, M137K, P138K, A139K, A141K, S142K, A143K, F144K, Q145K, R146K, R147K, S155K, H156K, Q158K, S159K, L161K, E162K, V163K, S164K, Y165K, R166K, V167K, L168K, R169K, H170K, L171K, A172K, Q173K and P174K.

Examples of preferred amino acid substitutions include one or more of Q70K, Q90K, T105K, Q120K, T133K, R146K, R147K, S159K, R166K and R169K.

The polypeptide conjugate of the invention having introduced and/or removed at least one lysine is preferably in vivo glycosylated, e.g. using naturally-occurring glycosylation sites present in the polypeptide. However, in a particular embodiment the conjugate is one wherein the amino acid sequence of the polypeptide differs from that of the native polypeptide in that at least one N-glycosylation site has been introduced and/or removed. Such introduced/removed sites may any of those described in the section entitled “Conjugate of the invention wherein the non-polypeptide moiety is an oligosaccharide moiety”.

ii) Removal of Lysine Residues

In order to avoid conjugation to one or more of the lysine residues present in one or more of the monomeric units (since these may inactivate or severely reduce the activity of the resulting conjugate if they are located in the receptor-binding domain), it may be desirable to remove at least one lysine residue. Accordingly, the conjugate according to this embodiment comprises at least one monomeric polypeptide unit comprising an amino acid sequence that differs from the native amino acid sequence in the removal of at least one lysine residue, in particular a lysine residue selected from those having more than 25% of their side chains surface exposed, preferably selected from those having more than 50% of their side chain surface exposed.

The removal is preferably achieved by substitution by any other amino acid residue, in particular an arginine or a glutamine residue.

hG-CSF contains four lysine residues, of which K16 is located in the receptor-binding domain and the others are located in positions 23, 34 and 40, respectively, all relatively close to the receptor-binding domain. Accordingly, one or both of the G-CSF monomeric units may comprise an amino acid sequence which, in addition to or instead of having one or more of the substitutions to lysine listed above, is modified in relation to hG-CSF by removal of at least one of the amino acid residues selected from the group consisting of K16, K23, K34 and K40, in particular at least K16, the removal preferably being achieved by substitution by any other amino acid residue, in particular an arginine or a glutamine residue. One or both of the subunits may thus have a single lysine residue removed, or all of the four native lysine residues removed, or two or three lysine residues removed, i.e. selected from the group consisting of: K16+K23; K16+K34; K16+K40; K16+K23+K34; K16+K23+K40; K16+K34+K40; K23+K34; K23+K40; K23+K34+K40; and K34+K40.

The single-chain G-CSF polypeptide according to this aspect of the invention preferably comprises at least one monomeric unit having at least one of the substitutions selected from the group consisting of K16R, K16Q, K23R, K23Q, K34R, K34Q, K40R and K40Q, more preferably at least one of the substitutions K16R and K23R, whereby conjugation of these residues can be avoided. Preferably, the polypeptide comprises at least one substitution selected from the group consisting of K16R+K23R, K16R+K34R, K16R+K40R, K23R+K34R, K23R+K40R, K34R+K40R, K16R+K23R+K34R, K16R+K23R+K40R, K23R+K34R+K40R and K16R+K34R+K40R. These substitutions are likely to give rise to the least structural difference.

In a preferred embodiment of this aspect of the invention, both of the monomeric units of the single-chain G-CSF polypeptide are modified by removal of one or more lysines as described above. The lysines that are removed may be the same in the two subunits or they may differ. If desired, removal of lysines in this embodiment may be accompanied by introduction of one or more lysines in one or both of the subunits, although it has been found that surprisingly advantageous results are obtained simply by removing one or more lysines from the two subunits, without any introduction of lysines, when lysine PEGylation is used.

iii) Introduction and Removal of Lysine Residues

In one embodiment the conjugate of the invention comprises at least one introduced lysine residue and at least one removed lysine residue.

Conjugate of the Invention Having a Non-lysine Residue as an Attachment Group

Based on the present disclosure the skilled person will be aware that amino acid residues comprising other attachment groups may be introduced into and/or removed from the native monomeric polypeptide using the same approach as that illustrated above with lysine residues. For instance, one or more amino acid residues comprising an acid group (glutamic acid or aspartic acid), asparagine, histidine, tyrosine or cysteine may be introduced into positions which are occupied by amino acid residues having surface exposed side chains, or removed (preferably by substitution by any other amino acid residue). Such modified polypeptides may further be in vivo glycosylated.

In the case of a single-chain G-CSF conjugate according to the invention, cysteine attachment groups may be provided by means of an amino acid sequence that differs, in at least one monomeric unit, from the amino acid sequence of hG-CSF shown in SEQ ID NO:1 in at least one substitution selected from the group consisting of T1C, P2C, L3C, G4C, P5C, A6C, S7C, S8C, L9C, P10C, Q11C, S12C, F13C, L14C, L15C, E19C, Q20C, V21C, R22C, Q25C, G26C, D27C, A29C, A30C, E33C, A37C, T38C, Y39C, L41C, H43C, P44C, E45C, E46C, V48C, L49C, L50C, H52C, S53C, L54C, I56C, P57C, P60C, L61C, S62C, S63C, P65C, S66C, Q67C, A68C, L69C, Q70C, L71C, A72C, G73C, S76C, Q77C, L78C, S80C, F83C, Q86C, G87C, Q90C, E93C, G94C, S96C, P97C, E98C, L99C, G100C, P101C, T102C, D104C, T105C, Q107C, L108C, D109C, A111C, D112C, F113C, T115C, T116C, W118C, Q119C, Q120C, M121C, E122C, E123C, L124C, M126C, A127C, P128C, A129C, L130C, Q131C, P132C, T133C, Q134C, G135C, A136C, M137C, P138C, A139C, A141C, S142C, A143C, F144C, Q145C, R146C, R147C, S155C, H156C, Q158C, S159C, L161C, E162C, V163C, S164C, Y165C, R166C, V167C, L168C, R169C, H170C, L171C, A172C, Q173C and P174C. The receptor-binding domain of hG-CSF contains a cysteine residue in position 17 which does not take part in a cystine and which may therefore advantageously be removed in order to avoid conjugate of a non-polypeptide moiety to said cysteine. Although C17 may be substituted by any other amino acid residue, it is in particular substituted by a serine residue.

Examples of preferred substitutions according to this aspect of the invention include R146C, R147C, R166C and R169C.

A single-chain G-CSF conjugate according to the invention may also have one or more non-polypeptide moieties bound to an acid group or to the C-terminal amino acid residue, in particular to an aspartic acid or glutamic acid residue. In this case, the amino acid sequence of at least one of the monomeric units may differ from the amino acid sequence shown in SEQ ID NO:1 in at least one substitution selected from the group consisting of T1D, P2D, L3D, G4D, P5D, A6D, S7D, S8D, L9D, P10D, Q11D, S12D, F13D, L14D, L15D, K16D, Q20D, V21D, R22D, K23D, Q25D, G26D, A29D, A30D, K34D, A37D, T38D, Y39D, K40D, L41D, H43D, P44D, V48D, L49D, L50D, H52D, S53D, L54D, I56D, P57D, P60D, L61D, S62D, S63D, P65D, S66D, Q67D, A68D, L69D, Q70D, L71D, A72D, G73D, S76D, Q77D, L78D, S80D, F83D, Q86D, G87D, Q90D, G94D, S96D, P97D, L99D, G100D, P101D, T102D, T105D, Q107D, L108D, A111D, F113D, T115D, T116D, W118D, Q119D, Q120D, M121D, L124D, M126D, A127D, P128D, A129D, L130D, Q131D, P132D, T133D, Q134D, G135D, A136D, M137D, P138D, A139D, A141D, S142D, A143D, F144D, Q145D, R146D, R147D, S155D, H156D, Q158D, S159D, L161D, V163D, S164D, Y165D, R166D, V167D, L168D, R169D, H170D, L171D, A172D, Q173D and P174D; or alternatively at least one substitution selected from the group consisting of T1E, P2E, L3E, G4E, P5E, A6E, S7E, S8E, L9E, P10E, Q11E, S12E, F13E, L14E, L15E, K16E, Q20E, V21E, R22E, K23E, Q25E, G26E, A29E, A30E, K34E, A37E, T38E, Y39E, K40E, L41E, H43E, P44E, V48E, L49E, L50E, H52E, S53E, L54E, 156E, P57E, P60E, L61E, S62E, S63E, P65E, S66E, Q67E, A68E, L69E, Q70E, L71E, A72E, G73E, S76E, Q77E, L78E, S80E, F83E, Q86E, G87E, Q90E, G94E, S96E, P97E, L99E, G100E, P101E, T102E, T105E, Q107E, L108E, A111E, F113E, T115E, T116E, W118E, Q119E, Q120E, M121E, L124E, M126E, A127E, P128E, A129E, L130E, Q131E, P132E, T133E, Q134E, G135E, A136E, M137E, P138E, A139E, A141E, S142E, A143E, F144E, Q145E, R146E, R147E, S155E, H156E, Q158E, S159E, L161E, V163E, S164E, Y165E, R166E, V167E, L168E, R169E, H170E, L171E, A172E, Q173E and P174E.

Examples of preferred substitutions according to this aspect of the invention include Q67E, Q70E, Q77E, Q86E, Q90E, Q120E, Q131E, Q134E, Q145E and Q173E.

In addition to one or more of the above listed substitutions to aspartic and/or glutamic acid, a G-CSF monomeric unit may by modified in relation to hG-CSF as shown in SEQ ID NO:1 by removal, preferably by substitution, of at least one of the amino acid residues selected from the group consisting of D27, D104, D109, D112, E19, E33, E45, E46, E93, E98, E122, E123 and E163. The substitution may be by any other amino acid residue, in particular by an asparagine or a glutamine residue, whereby conjugation of these residues to a non-polypeptide moiety can be avoided.

Conjugate of the Invention Wherein the Non-polypeptide Moiety is a Carbohydrate Moiety

In a further aspect the invention relates to a conjugate comprising a glycosylated polypeptide in which at least one non-naturally occurring glycosylation site has been introduced into the amino acid sequence.

A suitable N-glycosylation site may be introduced by introducing, preferably by substitution, an asparagine residue in a position occupied by an amino acid residue having more than 25% of its side chain exposed at the surface of the polypeptide, which position does not have a proline residue located in position +1 or +3 therefrom. If the amino acid residue located in position +2 is a serine or threonine, no further amino acid substitution is required. However, if this position is occupied by a different amino acid residue, a serine or threonine residue needs to be introduced.

In a dimeric G-CSF conjugate of the invention, one or more non-naturally occurring glycosylation sites may be introduced into at least one monomeric unit relative to the amino acid sequence of hG-CSF by way of at least one substitution selected from the group consisting of L3N+P5S/T, P5N, A6N, S8N+P10S/T, P10N, Q11N+F13S/T, S12N+L14S/T, F13N+L15S/T, L14N+K16S/T, K16N+L18S/T, E19N+V21S/T, Q20N+R22S/T, V21N+K23S/T, R22N+I24S/T, K23N+Q25S/T, Q25N+D27S/T, G26N+G28S/T, D27N+A29S/T, A29N+L31S/T, A30N+Q32S/T, E33N+L35S/T, A37N+Y39S/T, T38N+K40S/T, Y39N+L41S/T, P44N+E46S/T, E45N+L47S/T, E46N+V48S/T, V48N+L50S/T, L49N+G51S/T, L50N+H52S/T, H52N+L54S/T, S53N+G55S/T, P60N, L61N, S63N+P65S/T, P65N+Q67S/T, S66N+A68S/T, Q67N+L69S/T, A68N+Q70S/T, L69N+L71S/T, Q70N+A72S/T, L71N+G73S/T, G73N+L75S/T, S76N+L78S/T, Q77N+H79S/T, L78N, S80N+L82S/T, F83N+Y85S/T, Q86N+L88S/T, G87N+L89S/T,

Q90N+L92S/T, E93N+I95S/T, P97N+L99S/T, L99N+P101S/T, P101N+L103S/T, T102N+D 104S/T, D 104N+L106S/T, T105N+Q107S/T, Q107N+D109S/T, L108N+V101S/T, D109N+A111S/T, A111N+F113S/T, D112N+A114S/T, F113N, T115N+I117S/T, T116N+W118S/T, W118N+Q120S/T, Q119N+M121S/T, Q120N+E122S/T, M121N+E123S/T, E122N+L124S/T, E123N+G125S/T, L124N+M126S/T, M126N+P128S/T, P128N+L130S/T, L130N+P132S/T, P132N+Q134S/T, T133N+G135S/T, Q134N+A136S/T, A136N+P138S/T, P138N+F140S/T, A139N+A141S/T, A141N+A143S/T, S142N+F144S/T, A143N+Q145S/T, F144N+R146S/T, Q145N+R147S/T, R146N+A148S/T, R147N+G149S/T, S155N+L157S/T, H156N+Q158S/T, S159N+L161S/T, L161N+V163S/T, E162N, V163N+Y165S/T, S164N+R166S/T, Y165N+V167S/T, R166N+L168S/T, V167N+R169S/T, L168N+H170S/T, R169N+L171S/T and H170N+A172S/T, wherein S/T indicates an S or a T residue, preferably a T residue.

Alternatively, the conjugate according to this aspect may comprise at least one monomeric unit comprising an amino acid sequence that differs from that shown in SEQ ID NO :1 in at least one substitution selected from the group consisting of P5N, A6N, P10N, P60N, L61N, L78N, F113N and E162N, in particular from the group consisting of P5N, A6N, P10N, P60N, L61N, F113N and E162N, such as from the group consisting of P60N, L61N, F113N and E162N.

Alternatively, the conjugate according to this aspect may comprise at least one monomeric unit comprising an amino acid sequence that differs from that shown in SEQ ID NO:1 in at least one substitution selected from the group consisting of D27N+A29S, D27N+A29T, D104N+L106S, D104N+L106T, D109N+A111S, D109N+A111T, D112N+A114S and D112N+A114T, more preferably from the group consisting of D27N+A29S, D27N+A29T, D104N+L106S, D104N+L106T, D112N+A114S and D112N+A114T, such as from the group consisting of D27N+A29S, D27N+A29T, D104N+L106S and D104N+L106T.

Alternatively or additionally, the polypeptide may have an amino acid sequence in which at least one naturally occurring N-glycosylation site has been removed.

Furthermore, the amino acid sequence of a polypeptide having at least one of the above mentioned N-glycosylation site modifications may differ from the native sequence in that at least one lysine residue has been removed as identified above in the section entitled “Removal of lysine residues”.

It will be understood that in order to prepare a conjugate according to this aspect of the invention, the polypeptide must be expressed in a glycosylating host cell capable of attaching oligosaccharide moieties at the glycosylation sites or alternatively subjected to in vitro glycosylation. Examples of glycosylating host cells are given in the section below entitled “Coupling to an oligosaccharide moiety”.

In addition to a carbohydrate molecule, the conjugate according to the aspect of the invention described in the present section may contain additional non-polypeptide moieties, in particular a polymer molecule conjugated to one or more, optionally introduced attachment groups present in the polypeptide part of the conjugate.

Non-polypeptide Moiety of the Conjugate of the Invention

As indicated above, the non-polypeptide moiety of the conjugate of the invention is preferably selected from the group consisting of a polymer molecule, a lipophilic compound, an oligosaccharide moiety (e.g. by way of in vivo glycosylation) and an organic derivatizing agent. All of these moieties may confer desirable properties to the polypeptide part of the conjugate, in particular an increased functional in vivo half-life and/or an increased serum half-life. The polypeptide part of the conjugate is normally conjugated to only one type of non-polypeptide moiety, but it may also be conjugated to two or more different types of non-polypeptide moieties, e.g. to a polymer molecule and an oligosaccharide moiety, to a lipophilic group and an oligosaccharide moiety, to an organic derivatizing agent and an oligosaccharide moiety, to a lipophilic group and a polymer molecule, etc. The conjugation to two or more different non-polypeptide moieties may be done simultaneously or sequentially.

Methods for Preparing a Conjugate of the Invention

In the following sections “Conjugation to a lipophilic compound”, “Conjugation to a polymer molecule”, “Conjugation to an oligosaccharide moiety” and “Conjugation to an organic derivatizing agent”, conjugation to specific types of non-polypeptide moieties is described.

Conjugation to a Lipophilic Compound

The polypeptide and the lipophilic compound may be conjugated to each other either directly or by use of a linker. The lipophilic compound may be a natural compound such as a saturated or unsaturated fatty acid, a fatty acid diketone, a terpene, a prostaglandin, a vitamin, a carotenoid or steroid, or a synthetic compound such as a carbon acid, an alcohol, an amine or sulphonic acid with one or more alkyl, aryl, alkenyl or other multiple unsaturated compounds. The conjugation between the polypeptide and the lipophilic compound, optionally through a linker, may be done according to methods known in the art, e.g. as described by Bodanszky in Peptide Synthesis, John Wiley, New York, 1976 and in WO 96/12505.

Conjugation to a Polymer Molecule

The polymer molecule to be coupled to the polypeptide may be any suitable polymer molecule, such as a natural or synthetic homo-polymer or hetero-polymer, typically with a molecular weight in the range of 300-100,000 Da, such as 300-20,000 Da, more preferably in the range of 500-10,000 Da, such as in the range of 1000-5000 Da. Examples of homo-polymers include a polyol (i.e. poly-OH), a polyamine (i.e. poly-NH₂) and a polycarboxylic acid (i.e. poly-COOH). A hetero-polymer is a polymer which comprises different coupling groups, such as a hydroxyl group and an amine group.

Examples of suitable polymer molecules include polymer molecules selected from the group consisting of polyalkylene oxide (PAO), including polyalkylene glycol (PAG), such as polyethylene glycol (PEG) and polypropylene glycol (PPG), branched PEGs, poly-vinyl alcohol (PVA), poly-carboxylate, poly-(vinylpyrolidone), polyethylene-co-maleic acid anhydride, polystyrene-co-maleic acid anhydride, dextran, including carboxymethyl-dextran, or any other biopolymer suitable for reducing immunogenicity and/or increasing functional in vivo half-life and/or serum half-life. Another example of a polymer molecule is human albumin or another abundant plasma protein. Generally, polyalkylene glycol-derived polymers are biocompatible, non-toxic, non-antigenic, non-immunogenic, have various water solubility properties, and are easily excreted from living organisms.

PEG is the preferred polymer molecule, since it has only few reactive groups capable of cross-linking compared to e.g. polysaccharides such as dextran. In particular, mono-functional PEG, e.g. methoxypolyethylene glycol (mPEG), is of interest since its coupling chemistry is relatively simple (only one reactive group is available for conjugating with attachment groups on the polypeptide). Consequently, the risk of cross-linking is eliminated, the resulting polypeptide conjugates are more homogeneous and the reaction of the polymer molecules with the polypeptide is easier to control.

To effect covalent attachment of the polymer molecule(s) to the polypeptide, the hydroxyl end groups of the polymer molecule must be provided in activated form, i.e. with reactive functional groups. Suitable activated polymer molecules are commercially available, e.g. from Shearwater Corporation, Huntsville, Ala., USA. Alternatively, the polymer molecules can be activated by conventional methods known in the art, e.g. as disclosed in WO 90/13540. Specific examples of activated linear or branched polymer molecules for use in the present invention are described in the Shearwater Corp. 2001 Catalog (Polyethylene Glycol and Derivatives for Biomedical Applications, incorporated herein by reference). Specific examples of activated PEG polymers include the following linear PEGs: NHS-PEG (e.g. SPA-PEG, SSPA-PEG, SBA-PEG, SS-PEG, SSA-PEG, SC-PEG, SG-PEG, and SCM-PEG), and NOR-PEG), BTC-PEG, EPOX-PEG, NCO-PEG, NPC-PEG, CDI-PEG, ALD-PEG, TRES-PEG, VS-PEG, IODO-PEG, and MAL-PEG, and branched PEGs such as PEG2-NHS and those disclosed in U.S. Pat. Nos. 5,932,462 and 5,643,575, both of which are incorporated herein by reference. Furthermore, the following publications, incorporated herein by reference, disclose useful polymer molecules and/or PEGylation chemistries: U.S. Pat. Nos. 5,824,778, 5,476,653, WO 97/32607, EP 229,108, EP 402,378, U.S. Pat. Nos. 4,902,502, 5,281,698, 5,122,614, 5,219,564, WO 92/16555, WO 94/04193, WO 94/14758, WO 94/17039, WO 94/18247, WO 94/28024, WO 95/00162, WO 95/11924, WO 95/13090, WO 95/33490, WO 96/00080, WO 97/18832, WO 98/41562, WO 98/48837, WO 99/32134, WO 99/32139, WO 99/32140, WO 96/40791, WO 98/32466, WO 95/06058, EP 439 508, WO 97/03106, WO 96/21469, WO 95/13312, EP 921 131, U.S. Pat. No. 5,736,625, WO 98/05363, EP 809 996, U.S. Pat. No. 5,629,384, WO 96/41813, WO 96/07670, U.S. Pat. Nos. 5,473,034, 5,516,673, EP 605 963, U.S. Pat. No. 5,382,657, EP 510 356, EP 400 472, EP 183 503 and EP 154 316.

The conjugation of the polypeptide and the activated polymer molecules is conducted by use of any conventional method, e.g. as described in the following references (which also describe suitable methods for activation of polymer molecules): R. F. Taylor, (1991), “Protein immobilisation. Fundamental and applications”, Marcel Dekker, N. Y.; S. S. Wong, (1992), “Chemistry of Protein Conjugation and Crosslinking”, CRC Press, Florida, USA; G.T. Hermanson et al., (1993), “Immobilized Affinity Ligand Techniques”, Academic Press, N.Y.). The skilled person will be aware that the activation method and/or conjugation chemistry to be used depends on the attachment group(s) of the polypeptide (examples of which are given further above), as well as the functional groups of the polymer (e.g. amine, hydroxyl, carboxyl, aldehyde, sulfydryl, succinimidyl, maleimide, vinysulfone or haloacetate). The PEGylation may be directed towards conjugation to all available attachment groups on the polypeptide (i.e. such attachment groups that are exposed at the surface of the polypeptide) or may be directed towards one or more specific attachment groups, e.g. the N-terminal amino group (U.S. Pat. No. 5,985,265). Furthermore, the conjugation may be achieved in one step or in a stepwise manner (e.g. as described in WO 99/55377).

It will be understood that the PEGylation is designed so as to produce the optimal molecule with respect to the number of PEG molecules attached, the size and form of such molecules (e.g. whether they are linear or branched), and the attachment site(s) in the polypeptide. The molecular weight of the polymer to be used may e.g. be chosen on the basis of the desired effect to be achieved. For instance, if the primary purpose of the conjugation is to achieve a conjugate having a high molecular weight (e.g. to reduce renal clearance) it is usually desirable to conjugate as few high Mw polymer molecules as possible to obtain the desired molecular weight. When a high degree of epitope shielding is desirable this may be obtained by use of a sufficiently high number of low molecular weight polymer molecules (e.g. with a molecular weight of about 5000 Da) to effectively shield all or most epitopes of the polypeptide. For instance, 2-8, such as 3-6 such polymers may be used.

In connection with conjugation to only a single attachment group on the protein (as described in U.S. Pat. No. 5,985,265), it may be advantageous that the polymer molecule, which may be linear or branched, has a high molecular weight, e.g. about 20 kDa.

Normally, the polymer conjugation is performed under conditions aimed at reacting most or substantially all available polymer attachment groups with polymer molecules, in particular by using a molar excess of the non-polypeptide moiety relative to the polypeptide. Typically, the molar ratio of activated polymer molecules to polypeptide is at least about 5:1 and up to about 1000:1, e.g. in the range of from about 10:1 to about 200:1, in order to obtain optimal reaction.

It is also contemplated according to the invention to couple the polymer molecules to the polypeptide through a linker. Suitable linkers are well known to the skilled person. A preferred example is cyanuric chloride (Abuchowski et al., (1977), J. Biol. Chem., 252, 3578-3581; U.S. Pat. No. 4,179,337; Shafer et al., (1986), J. Polym. Sci. Polym. Chem., 24, 375-378).

Subsequent to the conjugation, residual activated polymer molecules are blocked according to methods known in the art, e.g. by addition of primary amine to the reaction mixture, and the resulting inactivated polymer molecules are removed by a suitable method.

In a specific embodiment, the polypeptide conjugate of the invention is one which comprises a single PEG molecule attached to the N-terminal of the polypeptide and no other PEG molecules, in particular a linear or branched PEG molecule with a molecular weight of at least about 20 kDa. The polypeptide according to this embodiment may further comprise one or more oligosaccharide moieties attached to an N-linked or O-linked glycosylation site of the polypeptide or carbohydrate moieties attached by in vitro glycosylation.

In another specific embodiment, the polypeptide conjugate of the invention comprises a PEG molecule attached to most or substantially all of the lysine residues in the polypeptide available for PEGylation, in particular a linear or branched PEG molecule, wherein each PEG e.g. has a molecular weight of about 5 kDa.

In yet another embodiment, the polypeptide conjugate of the invention comprises a PEG molecule attached to most or substantially all of the lysine residues in the polypeptide available for PEGylation, and in addition to the N-terminal amino acid residue of the polypeptide.

Covalent in vitro coupling of carbohydrate moiety glycosides (such as dextran) to amino acid residues of the polypeptide may also be used, e.g. as described in WO 87/05330 and in Aplin et al., CRC Crit. Rev. Biochem., pp. 259-306, 1981. The in vitro coupling of carbohydrate moieties or PEG to protein- and peptide-bound Gln residues can be carried out by transglutaminases (TGases). Transglutaminases catalyse the transfer of donor amine groups to protein- and peptide-bound Gln residues in a so-called cross-linking reaction. The donor-amine groups can be protein- or peptide-bound such as the ε-amino group in Lys-residues or can be part of a small or large organic molecule. An example of a small organic molecule functioning as amino donor in TGase-catalysed cross-linking is putrescine (1,4-diaminobutane). An example of a larger organic molecule functioning as amino donor in TGase-catalysed cross-linking is an amine-containing PEG (Sato et al., Biochemistry 35, 13072-13080).

TGases, in general, are highly specific enzymes, and not every Gln residue exposed on the surface of a protein is accessible to TGase-catalysed cross-linking to amino-containing substances. On the contrary, only a few Gln residues function naturally as TGase substrates, but the exact parameters governing which Gln residues are good TGase substrates remain unknown. Thus, in order to render a protein susceptible to TGase-catalysed cross-linking reactions it is often a prerequisite at convenient positions to add stretches of amino acid sequence known to function well as TGase substrates. Several amino acid sequences are known to be or to contain excellent natural TGase substrates e.g. substance P, elafin, fibrinogen, fibronectin, α₂-plasmin inhibitor, α-caseins, and β-caseins.

Coupling to an Oligosaccharide Moiety

The conjugation to an oligosaccharide moiety normally takes place by in vivo gly-cosylation effected by a glycosylating eucaryotic expression host. The expression host cell may be selected from fungal (filamentous fungal or yeast), insect or animal cells or from transgenic plant cells. In one embodiment the host cell is a mammalian cell, such as a CHO cell, a BHK or a HEK cell, e.g. HEK 293, an insect cell, such as an SF9 cell, or a yeast cell, e.g. Saccharomyces cerevisiae or Pichia pastoris, or any of the host cells mentioned herein-after. As indicated above, glycosylation may alternatively be performed in vitro by a method known per se in the art.

Coupling to an Organic Derivatizing Agent

Covalent modification of the polypeptide may be performed by reacting one or more attachment groups of the polypeptide with an organic derivatizing agent. Suitable derivatizing agents and methods are well known in the art. For example, cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β-(4-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole. Histidyl residues are derivatized by reaction with diethylpyrocarbonateat, pH 5.5-7.0, because this agent is relatively specific for the histidyl side chain. Para-bromophenacyl bromide is also useful. The reaction is preferably performed in 0.1 M sodium cacodylate at pH 6.0. Lysinyl and amino terminal residues are reacted with succinic or other carboxylic acid anhydrides. Derivatization with these agents has the effect of reversing the charge of the lysinyl residues. Other suitable reagents for derivatizing α-amino-containing residues include imidoesters such as methyl picolinimidate, pyridoxal phosphate, pyridoxal, chloroborohydride, trinitrobenzenesulfonic acid, O-methylisourea, 2,4-pentanedione and transaminase-catalyzed reaction with glyoxylate. Arginyl residues are modified by reaction with one or several conventional reagents, among them phenylglyoxal, 2,3-butanedione, 1,2-cyclohexanedione and ninhydrin. Derivatization of arginine residues requires that the reaction be performed in alkaline conditions because of the high pKa of the guanidine functional group.

Furthermore, these reagents may react with the groups of lysine as well as the arginine guanidino group. Carboxyl side groups (aspartyl or glutamyl) are selectively modified by reaction with carbodiimides (R—N═C═N—R′), where R and R′ are different alkyl groups, such as 1-cyclohexyl-3-(2-morpholinyl 4-ethyl) carbodiimide or 1-ethyl-3-(4-azonia-4,4-dimethylpentyl) carbodiiimide. Furthermore, aspartyl and glutamyl residues are converted to asparaginyl and glutaminyl residues by reaction with ammonium ions.

Conjugation of a Tagged Polypeptide

In an alternative embodiment the polypeptide is expressed as a fusion protein with a tag, i.e. an amino acid sequence or peptide stretch made up of typically 1-30, such as 1-20 amino acid residues. Besides allowing for fast and easy purification, the tag is a convenient tool for achieving conjugation between the tagged polypeptide and the non-polypeptide moiety. In particular, the tag may be used for achieving conjugation in microtiter plates or other carriers, such as paramagnetic beads, to which the tagged polypeptide can be immobilised via the tag. The conjugation to the tagged polypeptide in e.g. microtiter plates has the advantage that the tagged polypeptide can be immobilised in the microtiter plates directly from the culture broth (in principle without any purification) and subjected to conjugation. Thereby, the total number of process steps (from expression to conjugation) can be reduced. Furthermore, the tag may function as a spacer molecule ensuring an improved accessibility to the immobilised polypeptide to be conjugated. The conjugation using a tagged polypeptide may be to any of the non-polypeptide moieties disclosed herein, e.g. to a polymer molecule such as PEG.

The identity of the specific tag to be used is not critical as long as the tag is capable of being expressed with the polypeptide and is capable of being immobilised on a suitable surface or carrier material. A number of suitable tags are commercially available, e.g. from Unizyme Laboratories, Denmark. For instance, the tag may consist of any of the following sequences (SEQ ID NOS: 9-13, respectively):

His-His-His-His-His-His

Met-Lys-His-His-His-His-His-His

Met-Lys-His-His-Ala-His-His-Gln-His-His

Met-Lys-His-Gln-His-Gln-His-Gln-His-Gln-His-Gln-His-Gln

Met-Lys-His-Gln-His-Gln-His-Gln-His-Gln-His-Gln-His-Gln-Gln

or any of (SEQ ID NOS: 14-16, respectively):

EQKLI SEEDL (a C-terminal tag described in Mol. Cell. Biol. 5:3610-16, 1985)

DYKDDDDK (a C- or N-terminal tag)

YPYDVPDYA

Antibodies against the above tags are commercially available, e.g. from Alpha Diagnostic International, Inc., USA, and from Aves Lab, Inc., USA.

A convenient method for using a tagged polypeptide for PEGylation is given in the Materials and Methods section below. The subsequent cleavage of the tag from the polypeptide may be achieved by use of commercially available enzymes.

Methods for Preparing the Polypeptide of the Conjugate of the Invention

The polypeptide of the present invention or the polypeptide part of a conjugate of the invention, optionally in glycosylated form, may be produced by any suitable method known in the art. Such methods include constructing a nucleotide sequence encoding the polypeptide and expressing the sequence in a suitable transformed or transfected host. However, polypeptides of the invention may be produced, albeit less efficiently, by chemical synthesis or a combination of chemical synthesis or a combination of chemical synthesis and recombinant DNA technology.

The invention thus encompasses a method for preparing a single-chain multimeric polypeptide or polypeptide conjugate as disclosed herein, comprising culturing a recombinant host cell comprising a single nucleotide sequence encoding said polypeptide in a suitable culture medium under conditions permitting expression of the nucleotide sequence, and recovering the resulting polypeptide from the cell culture, followed, where appropriate, by reacting the polypeptide with a polymer molecule or other non-polypeptide moiety under conditions permitting conjugation to take place so as to result in a polypeptide conjugate, and recovering the conjugate.

The nucleotide sequence encoding a multimeric polypeptide of the invention, or the polypeptide part of a conjugate of the invention, may be constructed by isolating or synthesizing a nucleotide sequence encoding the parent polypeptide, and then changing the nucleotide sequence so as to effect introduction (i.e. insertion or substitution) or deletion (i.e. removal or substitution) of the relevant amino acid residue(s). The nucleotide sequence may be conveniently modified by site-directed mutagenesis in accordance with conventional methods. Alternatively, the nucleotide sequence may be prepared by chemical synthesis, e.g. by using an oligonucleotide synthesizer, wherein oligonucleotides are designed based on the amino acid sequence of the desired polypeptide, and preferably selecting those codons that are favored in the host cell in which the recombinant polypeptide will be produced. For example, several small oligonucleotides coding for portions of the desired polypeptide may be synthesized and assembled by PCR, ligation or ligation chain reaction (LCR) (Barany, PNAS 88:189-193, 1991). The individual oligonucleotides typically contain 5′ or 3′ over-hangs for complementary assembly.

Suitable mutations may be introduced by, e.g., site-directed mutagenesis as described by Sambrook et al. (Molecular Cloning: A Laboratory Manual, Second Edition, 1989), or by random mutagenesis or DNA shuffling, e.g. as described below followed by screening for sequences coding for polypeptides with the desired activity. Screening may be carried out by an assay method as described below.

Random mutagenesis (whether performed in the whole nucleotide sequence or one or more selected regions thereof) may be performed by any suitable method. For example, random mutagenesis is performed using a suitable physical or chemical mutagenizing agent, a suitable oligonucleotide, PCR generated mutagenesis or any combination of these mutagenizing agents/methods according to state of the art technology, e.g. as disclosed in

Error prone PCR generated mutagenesis, e.g. as described by J. O. Deshler (1992), GATA 9(4): 103-106 and Leung et al., Technique (1989) Vol. 1, No. 1, pp. 11-15, is particularly useful for mutagenesis of longer peptide stretches (corresponding to nucleotide sequences containing more than 100 bp) or entire genes, and is preferably performed under conditions that increase the misincorporation of nucleotides.

Random mutagenesis based on doped or spiked oligonucleotides is of particular use for mutagenesis of one or more regions containing shorter nucleotide sequences (normally containing less than 100 nucleotides per region). Mutagenesis of several regions is conveniently conducted by using several doped oligonucleotides and combining them by PCR. Doped or spiked oligonucleotides may also be used for random mutagenesis of nucleotide sequences encoding longer peptide stretches or entire genes when it is desirable to be able to control the random mutagenesis to a higher extent than what is possible with error prone PCR generated mutagenesis.

Conveniently, random mutagenesis of one or more selected regions of a nucleotide sequence encoding the polypeptide of interest is performed using PCR generated mutagenesis, in which one or more suitable oligonucleotide probes which flank the area to be mutagenized are used. Preferably, for mutagenesis of selected peptide stretches doped or spiked oligonucleotides is used. The doping or spiking can be designed to introduce any kind of amino acid residue and/or to avoid a codon for an unwanted amino acid residue (by lowering the amount of or completely avoiding the nucleotides resulting in this codon). The doping may be designed on the basis of the skilled person's intelligent consideration of nucleotide doping (in accordance with generally known principles), by use of a suitable algorithm, e.g. a computer program which is based on the algorithm described by Siderovski D P and Mak T W, Comput. Biol. Med. (1993) Vol. 23, No. 6, pp. 463-474 or Jensen et al. Nucleic Acids Research, 1998, Vol. 26, No. 3 or by using trinucleotides (Sondek, J. and Shortle, D., Proc. Natl. Acad. Sci, USA, Vol. 89, pp. 3581-3585, April 1992; Kayushin et al., Nucleic Acids Research, 1996, Vol. 24, No. 19, pp. 3748-3755; Virnekäs et al., Nucleic Acids Research, 1994, Vol. 22, No. 25; WO 93/21203). The doped or spiked oligonucleotide can be incorporated into the nucleotide sequence encoding the polypeptide of interest by any published technique using e.g. PCR, LCR or any DNA polymerase or ligase.

Random mutagenesis may be performed in two, three, four, five, six or more regions at the same time by synthesizing doped oligonucleotides covering each region and assembling the oligonucleotides by state of the art technologies, for example by a PCR method. One convenient PCR method involves a PCR reaction wherein the nucleotide sequence encoding the polypeptide of interest is used as a template and the doped oligonucleotides are used as primers. In addition, cloning primers localized outside the targeted regions may be used. The resulting PCR product can either be directly cloned into an appropriate expression vector or gel purified and amplified in a second PCR reaction using the cloning primers and cloned into an appropriate expression vector.

Besides substitutions the random mutagenesis may also cover random introduction of insertions or deletions. Preferably, the insertions are made so as to be in reading frame, e.g. by performing multiple introduction of three nucleotides as described by Hallet et al., Nucleic Acids Res. 1997, 25(9):1866-7 and Sondek and Shortle, PNAS USA 1992, 89(8):3581-5.

The nucleotide sequence(s) or nucleotide sequence region(s) to be mutagenized are typically present on a suitable vector such as a plasmid or a bacteriophage, which as such is incubated with or otherwise exposed to the mutagenizing agent. The nucleotide sequence(s) to be mutagenized may also be present in a host cell either by being integrated into the genome of said cell or by being present on a vector harboured in the cell. Alternatively, the nucleotide sequence to be mutagenized is in isolated form. The nucleotide sequence is preferably a DNA sequence such as a cDNA, genomic DNA or synthetic DNA sequence.

In one embodiment the random mutagenesis is accompanied by conjugation to a non-polypeptide moiety. More specifically, a modified conjugated single-chain polypeptide of the invention may be prepared by

a) expressing a random mutagenized library of nucleotide sequences encoding a parent polypeptide in single-chain form,

b) conjugating one or more non-polypeptide moieties to the polypeptide variants expressed in step a),

c) screening the resulting conjugates for agonist activity or receptor-binding,

d) selecting polypeptide conjugates having such capability, and

e) optionally subjecting the nucleotide sequence encoding the polypeptide part of a polypeptide conjugate selected in step d) to one or more repeated cycles of steps a)-d).

The above method for random mutagenesis and conjugation is further described in PCT/DK00/00371.

When using random mutagenesis as outlined above, the expression step a) can be conducted in any suitable manner, and conveniently as described further below. Suitably, the random mutagenized library is prepared by subjecting a nucleotide sequence encoding the parent polypeptide in single-chain form to random mutagenesis so as to create a large number of mutated nucleotide sequences. The random mutagenesis may be entirely random, both with respect to where in the nucleotide sequence the mutagenesis occurs and with respect to the nature of mutagenesis.

Alternatively, the random mutagenesis may be conducted so as to randomly mutate one or more selected regions of the polypeptide, in particular a receptor-binding site thereof. The library is typically present in a host cell, from which expression is achieved. Of particular interest is a host cell which is capable of a reasonable transformation frequency such as bacterium, e.g. E. coli, yeast, e.g. S. cereviciae, or fungus. Alternatively, a high throughput transfection system of mammalian cells or other cells capable of a desirable post-translational modification (such as in vivo glycosylation) may be employed, for example using CHO (Chinese Hamster Ovary), COS or BHK (Baby Hamster Kidney) cells.

Conjugation step b) is conveniently conducted as described above in connection with conjugation to a polymer or an oligosaccharide moiety. The screening step c) is an important element of the method according to this embodiment of the invention. The screening is conveniently conducted as a primary screening for activating or receptor-binding capability, e.g. based on the principles disclosed in the section entitled “Assay”.

In a preferred embodiment as many as possible of steps a-d) are performed in a high throughput screening system. In particular, it is preferred that steps a)-d) are performed in a robotized system, wherein the expression from the random mutagenized library of nucleotide sequences is achieved in microtiter plates, the resulting supernatant is transferred to a different microtiter plate, preferably under conditions allowing immobilization of the polypeptides, and optionally under conditions where the receptor-binding site of the polypeptide is blocked, e.g. by a suitable receptor, receptor analogue or antibody, and/or the polypeptide is provided with a tag, e.g. of the type described above. The optionally immobilized, blocked and/or tagged polypeptides are subjected to conjugation to a non-polypeptide moiety while present in the microtiter plate and the resulting polypeptide conjugates present in the microtiter plate are subjected to the relevant screening. Subsequently, selected positive polypeptide conjugates are subjected to further characterization, including secondary screening.

Once assembled, the nucleotide sequence encoding the polypeptide is inserted into a recombinant vector and operably linked to control sequences necessary for expression of the polypeptide in the desired transformed host cell.

In a preferred embodiment the polypeptide conjugate can be prepared in a high throughput screening system allowing production and screening of a high number of different polypeptides in a short time. This is in particular suitable in the following situations:

obtaining an improved binding affinity

altering receptor specificity

reducing/eliminating possible antagonist activity

identifying optimal linkers.

Nucleotide sequence modification methods suitable for producing polypeptide variants for high throughput screening further include for instance methods which involve homologous cross-over such as disclosed in U.S. Pat. No. 5,093,257, and methods which involve gene shuffling, i.e. recombination between two or more homologous nucleotide sequences resulting in new nucleotide sequences having a number of nucleotide alterations when compared to the starting nucleotide sequences. Gene shuffling (also known as DNA shuffling) involves one or more cycles of random fragmentation and reassembly of the nucleotide sequences, followed by screening to select nucleotide sequences encoding polypeptides with desired properties. In order for homology-based nucleic acid shuffling to take place, the relevant parts of the nucleotide sequences are preferably at least 50% identical, such as at least 60% identical, more preferably at least 70% identical, such as at least 80% identical. The recombination can be performed in vitro or in vivo.

Examples of suitable in vitro gene shuffling methods are disclosed by Stemmer et al. (1994), Proc. Natl. Acad. Sci. USA; vol. 91, pp. 10747-10751; Stemmer (1994), Nature, vol. 370, pp. 389-391; Smith (1994), Nature vol. 370, pp. 324-325; Zhao et al., Nat. Biotechnol. March, 1998; 16(3): 258-61; Zhao H. and Arnold, F B, Nucleic Acids Research, 1997, Vol. 25. No. 6 pp. 1307-1308; Shao et al., Nucleic Acids Research Jan. 15, 1998; 26(2): pp. 681-83; and WO 95/17413. An example of a suitable in vivo shuffling method is disclosed in WO 97/07205. Other techniques for mutagenesis of nucleic acid sequences by in vitro or in vivo recombination are disclosed e.g. in WO 97/20078 and U.S. Pat. No. 5,837,458. Examples of specific shuffling techniques include “family shuffling”, “synthetic shuffling” and “in silico shuffling”. Family shuffling involves subjecting a family of homologous genes from different species to one or more cycles of shuffling and subsequent screening or selection. Family shuffling techniques are disclosed e.g. by Crameri et al. (1998), Nature, vol. 391, pp. 288-291; Christians et al. (1999), Nature Biotechnology, vol. 17, pp. 259-264; Chang et al. (1999), Nature Biotechnology, vol. 17, pp. 793-797; and Ness et al. (1999), Nature Biotechnology, vol. 17, 893-896. Synthetic shuffling involves providing libraries of overlapping synthetic oligonucleotides based e.g. on a sequence alignment of homologous genes of interest. The synthetically generated oligonucleotides are recombined, and the resulting recombinant nucleic acid sequences are screened and if desired used for further shuffling cycles. Synthetic shuffling techniques are disclosed in WO 00/42561. In silico shuffling refers to a DNA shuffling procedure which is performed or modelled using a computer system, thereby partly or entirely avoiding the need for physically manipulating nucleic acids. Techniques for in silico shuffling are disclosed in WO 00/42560.

It should of course be understood that not all vectors and expression control sequences function equally well to express the nucleotide sequence encoding a polypeptide described herein. Neither will all hosts function equally well with the same expression system. However, one skilled in the art will be able to make a selection among these vectors, expression control sequences and hosts without undue experimentation. For example, in selecting a vector, the host must be considered because the vector must replicate in it or be able to integrate into the chromosome. The vector's copy number, the ability to control that copy number, and the expression of any other proteins encoded by the vector, such as antibiotic markers, should also be considered. In selecting an expression control sequence, a variety of factors should also be considered. These include, for example, the relative strength of the sequence, its controllability, and its compatibility with the nucleotide sequence encoding the polypeptide, particularly as regards potential secondary structures. Hosts should be selected by consideration of their compatibility with the chosen vector, the toxicity of the product coded for by the nucleotide sequence, their secretion characteristics, their ability to fold the polypeptide correctly, their fermentation or culture requirements, and ease of purification of the products encoded by the nucleotide sequence.

The recombinant vector may be an autonomously replicating vector, i.e. a vector which exists as an extrachromosomal entity, the replication of which is independent of chromosomal replication, e.g. a plasmid. Alternatively, the vector is one which, when introduced into a host cell, is integrated into the host cell genome and replicated together with the chromosome(s) into which it has been integrated.

The vector is preferably an expression vector in which the nucleotide sequence encoding the polypeptide of the invention is operably linked to additional segments required for transcription of the nucleotide sequence. The vector is typically derived from plasmid or viral DNA. A number of suitable expression vectors for expression in the host cells mentioned herein are commercially available or described in the literature. Useful expression vectors for eukaryotic hosts include, for example, vectors comprising expression control sequences from SV40, bovine papilloma virus, adenovirus and cytomegalovirus. Specific vectors are, e.g., pCDNA3.1(+)\Hyg (Invitrogen, Carlsbad, Calif., USA) and pCI-neo (Stratagene, La Jolla, Calif., USA). Useful expression vectors for yeast cells include the 2μ plasmid and derivatives thereof, the POT1 vector (U.S. Pat. No. 4,931,373), the pJSO37 vector described in Okkels, Ann. New York Acad. Sci. 782, 202-207, 1996, and pPICZ A, B or C (Invitrogen). Useful vectors for insect cells include pVL941, pBG311 (Cate et al., “Isolation of the Bovine and Human Genes for Mullerian Inhibiting Substance and Expression of the Human Gene in Animal Cells”, Cell, 45, pp. 685-98, 1986), pBluebac 4.5 and pMelbac (both available from Invitrogen). Useful expression vectors for bacterial hosts include known bacterial plasmids, such as plasmids from E. coli, including pBR322, pET3a and pET12a (both from Novagen Inc., Wis., USA), wider host range plasmids, such as RP4, phage DNAs, e.g. the numerous derivatives of phage lambda, e.g. NM989, and other DNA phages, such as M13 and filamentous single stranded DNA phages.

Other vectors for use in this invention include those that allow the nucleotide sequence encoding the polypeptide to be amplified in copy number. Such amplifiable vectors are well known in the art. They include, for example, vectors able to be amplified by DHFR amplification (see, e.g., Kaufman, U.S. Pat. No. 4,470,461, Kaufman and Sharp, “Construction Of A Modular Dihydrafolate Reductase cDNA Gene: Analysis Of Signals Utilized For Efficient Expression”, Mol. Cell Biol., 2, pp. 1304-19 (1982)) and glutamine synthetase (“GS”) amplification (see e.g. U.S. Pat. No. 5,122,464 and EP 338,841).

The recombinant vector may further comprise a DNA sequence enabling the vector to replicate in the host cell in question. An example of such a sequence (when the host cell is a mammalian cell) is the SV40 origin of replication. When the host cell is a yeast cell, suitable sequences enabling the vector to replicate are the yeast plasmid 2μ replication genes REP 1-3 and origin of replication.

The vector may also comprise a selectable marker, e.g. a gene whose product complements a defect in the host cell, such as the gene coding for dihydrofolate reductase (DHFR) or the Schizosaccharomyces pombe TPI gene (described by P. R. Russell, Gene 40, 1985, pp. 125-130), or one which confers resistance to a drug, e.g. ampicillin, kanamycin, tetracyclin, chloramphenicol, neomycin, hygromycin or methotrexate. For S. cerevisiae, selectable markers include ura3 and leu2. For filamentous fungi, selectable markers include amdS, pyrG, arcB, niaD and sC.

The term “control sequences” is defined herein to include all components that are necessary or advantageous for the expression of the polypeptide of the invention. Each control sequence may be native or foreign to the nucleic acid sequence encoding the polypeptide. Such control sequences include, but are not limited to, a leader sequence, polyadenylation sequence, propeptide sequence, promoter, enhancer or upstream activating sequence, signal peptide sequence, and transcription terminator. At a minimum, the control sequences include a promoter.

A wide variety of expression control sequences may be used in the present invention. Such useful expression control sequences include the expression control sequences associated with structural genes of the foregoing expression vectors as well as any sequence known to control the expression of genes of prokaryotic or eukaryotic cells or their viruses, and various combinations thereof.

Examples of suitable control sequences for directing transcription in mammalian cells include the early and late promoters of SV40 and adenovirus, e.g. the adenovirus 2 major late promoter, the MT-1 (metallothionein gene) promoter, the human cytomegalovirus immediate-early gene promoter (CMV), the human elongation factor 1α (EF-1α) promoter, the Drosophila minimal heat shock protein 70 promoter, the Rous Sarcoma Virus (RSV) promoter, the human ubiquitin C (UbC) promoter, the human growth hormone terminator, SV40 or adenovirus Elb region polyadenylation signals and the Kozak consensus sequence (Kozak, M. J Mol Biol Aug. 20, 1987;196(4):947-50).

In order to improve expression in mammalian cells a synthetic intron may be inserted in the 5′ untranslated region of the nucleotide sequence encoding the polypeptide. An example of a synthetic intron is the synthetic intron from the plasmid pCI-Neo (available from Promega Corporation, Wis., USA).

Examples of suitable control sequences for directing transcription in insect cells include the polyhedrin promoter, the P10 promoter, the Autographa californica polyhedrosis virus basic protein promoter, the baculovirus immediate early gene 1 promoter, the baculovirus 39K delayed-early gene promoter, and the SV40 polyadenylation sequence. Examples of suitable control sequences for use in yeast host cells include the promoters of the yeast α-mating system, the yeast triose phosphate isomerase (TPI) promoter, promoters from yeast glycolytic genes or alcohol dehydrogenase genes, the ADH2-4c promoter, and the inducible GAL promoter. Examples of suitable control sequences for use in filamentous fungal host cells include the ADH3 promoter and terminator, a promoter derived from the genes encoding Aspergillus oryzae TAKA amylase triose phosphate isomerase or alkaline protease, an A. niger α-amylase, A. niger or A. nidulans glucoamylase, A. nidulans acetamidase, Rhizomucor miehei aspartic proteinase or lipase, the TPI1 terminator and the ADH3 terminator. Examples of suitable control sequences for use in bacterial host cells include promoters of the lac system, the trp system, the TAC or TRC system, and the major promoter regions of phage lambda.

The presence or absence of a signal peptide will e.g. depend on the expression host cell used for the production of the polypeptide to be expressed (whether it is an intracellular or extracellular polypeptide) and whether it is desirable to obtain secretion. For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide is preferably derived from a gene encoding A. oryzae TAKA amylase, A. niger neutral α-amylase, A. niger acid-stable amylase, or A. niger glucoamylase. For use in insect cells, the signal peptide may conveniently be derived from an insect gene (cf. WO 90/05783), such as the Lepidopteran manduca sexta adipokinetic hormone precursor, (cf. U.S. Pat. No. 5,023,328), the honeybee melittin (Invitrogen), ecdysteroid UDPglucosyltransferase (egt) (Murphy et al., Protein Expression and Purification 4, 349-357 (1993) or human pancreatic lipase (hp1) (Methods in Enzymology 284, pp. 262-272, 1997). A preferred signal peptide for use in mammalian cells is e.g. the murine Ig kappa light chain signal peptide (Coloma, M (1992) J. Imm. Methods 152:89-104). For use in yeast cells, suitable signal peptides have been found to be the α-factor signal peptide from S. cereviciae (cf. U.S. Pat. No. 4,870,008), a modified carboxypeptidase signal peptide (cf. L. A. Valls et al., Cell 48, 1987, pp. 887-897), the yeast BARI signal peptide (cf. WO 87/02670), the yeast aspartic protease 3 (YAP3) signal peptide (cf M. Egel-Mitani et al., Yeast 6, 1990, pp. 127-137), and the synthetic leader sequence TA57 (WO98/32867). For use in E. coli cells a suitable signal peptide has been found to be the signal peptide ompA (EP 581 821).

The nucleotide sequence of the invention encoding a polypeptide, whether prepared by site-directed mutagenesis, synthesis, PCR or other methods, may optionally also include a nucleotide sequence that encodes a signal peptide. The signal peptide is present when the polypeptide is to be secreted from the cells in which it is expressed. Such a signal peptide, if present, should be one recognized by the cell chosen for expression of the polypeptide. The signal peptide may be homologous (e.g. be that normally associated with the native peptide) or heterologous (i.e. originating from another source) to the polypeptide or may be homologous or heterologous to the host cell, i.e. be a signal peptide normally expressed from the host cell or one which is not normally expressed from the host cell. Accordingly, the signal peptide may be prokaryotic, e.g. derived from a bacterium such as E. coli, or eukaryotic, e.g. derived from a mammalian, insect or yeast cell.

Any suitable host may be used to produce the polypeptide or polypeptide part of the conjugate of the invention, including bacteria, fungi (including yeasts), plants, insects, mammals or other animals, or an appropriate animal cell line or another cell line. Examples of bacterial host cells include gram-positive bacteria such as strains of Bacillus, e.g. B. brevis or B. subtilis, Pseudomonas or Streptomyces, or gram-negative bacteria such as strains of E. coli. The introduction of a vector into a bacterial host cell may, for instance, be effected by protoplast transformation (see e.g. Chang and Cohen, 1979, Molecular General Genetics 168: 111-115), using competent cells (see e.g. Young and Spizizin, 1961, Journal of Bacteriology 81: 823-829, or Dubnau and Davidoff-Abelson, 1971, Journal of Molecular Biology 56: 209-221), electroporation (see e.g. Shigekawa and Dower, 1988, Biotechniques 6: 742-751), or conjugation (see e.g. Koehler and Thorne, 1987, Journal of Bacteriology 169: 5771-5278).

Examples of suitable filamentous fungal host cells include strains of Aspergillus, e.g. A. oryzae, A. niger or A. nidulans, Fusarium or Trichoderma. Fungal cells may be transformed by a process involving protoplast formation, transformation of the protoplasts, and regeneration of the cell wall in a manner known per se. Suitable procedures for transformation of Aspergillus host cells are described in EP 238 023 and U.S. Pat. No. 5,679,543. Suitable methods for transforming Fusarium species are described by Malardier et al., 1989, Gene 78: 147-156 and WO 96/00787. Examples of suitable yeast host cells include strains of Saccharomyces, e.g. S. cerevisiae, Schizosaccharomyces, Klyveromyces, Pichia, such as P. pastoris or P. methanolica, Hansenula, such as H. polymorpha, or Yarrowia. Yeast may be transformed using the procedures described by Becker and Guarente, In Abelson, J. N. and Simon, M. I., editors, Guide to Yeast Genetics and Molecular Biology, Methods in Enzymology, Volume 194, pp. 182-187, Academic Press, Inc., New York; Ito et al., 1983, Journal of Bacteriology 153: 163; Hinnen et al., 1978, PNAS USA 75: 1920: and as disclosed by Clontech Laboratories, Inc., Palo Alto, Calif., USA (in the product protocol for the Yeastmaker™ Yeast Transformation System Kit).

Examples of suitable insect host cells include a Lepidoptora cell line, such as Spodoptera frugiperda (Sf9 or Sf21) or Trichoplusioa ni cells (High Five) (U.S. Pat. No. 5,077,214). Transformation of insect cells and production of heterologous polypeptides therein may be performed as described by Invitrogen. Examples of suitable mammalian host cells include Chinese hamster ovary (CHO) cell lines, (e.g. CHO-K1; ATCC CCL-61), Green Monkey cell lines (COS) (e.g. COS 1 (ATCC CRL-1650), COS 7 (ATCC CRL-1651)); mouse cells (e.g. NS/O), Baby Hamster Kidney (BHK) cell lines (e.g. ATCC CRL-1632 or ATCC CCL-10), and human cells (e.g. HEK 293 (ATCC CRL-1573)), as well as plant cells in tissue culture.

Additional suitable cell lines are known in the art and available from public depositories such as the American Type Culture Collection, Rockville, Md., USA. Methods for introducing exogeneous DNA into mammalian host cells include calcium phosphate-mediated transfection, electroporation, DEAE-dextran mediated transfection, liposome-mediated transfection, viral vectors and the transfection method described by Life Technologies Ltd, Paisley, UK using Lipofectamin 2000. These methods are well known in the art and e.g. described by Ausbel et al. (eds.), 1996, Current Protocols in Molecular Biology, John Wiley & Sons, New York, USA. The cultivation of mammalian cells is conducted according to established methods, e.g. as disclosed in: Animal Cell Biotechnology, Methods and Protocols, Edited by Nigel Jenkins, 1999, Human Press Inc., Totowa, N.J., USA, and Harrison M A and Rae I F, General Techniques of Cell Culture, Cambridge University Press, 1997.

In the production methods of the present invention, the cells are cultivated in a nutrient medium suitable for production of the polypeptide using methods known in the art. For example, the cell may be cultivated by shake flask cultivation, small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g. in catalogues of the American Type Culture Collection). If the polypeptide is secreted into the nutrient medium, the polypeptide can be recovered directly from the medium. If the polypeptide is not secreted, it can be recovered from cell lysates.

The resulting polypeptide may be recovered by methods known in the art. For example, the polypeptide may be recovered from the nutrient medium by conventional procedures including, but not limited to, centrifugation, filtration, extraction, spray drying, evaporation or precipitation.

The polypeptides may be purified by a variety of procedures known in the art including, but not limited to, chromatography (e.g. ion exchange, affinity, hydrophobic, chromatofocusing, and size exclusion), electrophoretic procedures (e.g. preparative isoelectric focusing), differential solubility (e.g. ammonium sulfate precipitation), SDS-PAGE, or extraction (see e.g. Protein Purification, J.-C. Janson and Lars Ryden, editors, VCH Publishers, New York, 1989). Specific methods for purifying cytokine polypeptides are described in Human Cytokines, Handbook of Basic and Clinical Research, Volume II, Blackwell Science, Eds. Aggarwal and Gutterman, 1996, pp. 19-42.

Pharmaceutical Use and Formulations

The multimeric polypeptides and conjugates of the invention may be used for the manufacture of a medicament for treatment of diseases in mammals, in particular humans. The exact dose of a particular polypeptide or conjugate to be administered will depend on e.g. the disease, the administration schedule, whether it is administered alone or in conjunction with other therapeutic agents, the serum half-life of the compositions, and the general health of the patient. The polypeptide or conjugate will advantageously be administered in an “effective amount”, meaning an amount that prevents or alleviates, to any degree, or eliminates, the condition being treated.

Pharmaceutical formulations of the polypeptide or conjugate of the invention are typically administered in a composition that includes one or more pharmaceutically acceptable carriers or excipients. Such pharmaceutical compositions may be prepared in a manner known per se in the art to result in a polypeptide pharmaceutical that is sufficiently storage-stable and is suitable for administration to humans or animals.

Drug Form

The polypeptide or conjugate of the invention can be used “as is” and/or in a salt form thereof. Suitable salts include, but are not limited to, salts with alkali metals or alkaline earth metals, such as sodium, potassium, calcium and magnesium, as well as e.g. zinc salts. These salts or complexes may by present as a crystalline and/or amorphous structure.

Excipients

“Pharmaceutically acceptable” means a carrier or excipient that at the dosages and concentrations employed does not cause any untoward effects in the patients to whom it is administered. Such pharmaceutically acceptable carriers and excipients are well known in the art (see Remington's Pharmaceutical Sciences, 18th edition, A. R. Gennaro, Ed., Mack Publishing Company [1990]; Pharmaceutical Formulation Development of Peptides and Proteins, S. Frokjaer and L. Hovgaard, Eds., Taylor & Francis [2000]; and Handbook of Pharmaceutical Excipients, 3rd edition, A. Kibbe, Ed., Pharmaceutical Press [2000]).

Dose

The polypeptides and conjugates of the invention will be administered to patients in a therapeutically effective dose. By “therapeutically effective dose” herein is meant a dose that is sufficient to produced the desired effects in relation to the condition for which it is administered. The exact dose will depend on the disorder to be treated, and will be ascertainable by one skilled in the art using known techniques.

Mix of drugs

The pharmaceutical composition of the invention may be administered alone or in conjunction with other therapeutic agents. These agents may be incorporated as part of the same pharmaceutical composition or may be administered separately from the polypeptide or conjugate of the invention, either concurrently or in accordance with another treatment schedule. In addition, the polypeptide, conjugate or pharmaceutical composition of the invention may be used as an adjuvant to other therapies.

Patients

A “patient” for the purposes of the present invention includes both humans and other mammals. Thus the methods are applicable to both human therapy and veterinary applications.

Types of Composition and Administration Route

The pharmaceutical composition of the polypeptide or conjugate of the invention may be formulated in a variety of forms, e.g. as a liquid, gel, lyophilized, or as a compressed solid. The preferred form will depend upon the particular indication being treated and will be readily able to be determined by one skilled in the art.

The administration of the formulations of the present invention can be performed in a variety of ways, including, but not limited to, subcutaneously, intravenously, orally, intracerebrally, intranasally, transdermally, intraperitoneally, intramuscularly, intrapulmonary, vaginally, rectally, intraocularly, or in any other acceptable manner. The formulations can be administered continuously by infusion, although bolus injection is acceptable, using techniques well known in the art, such as pumps or implantation.

Parenterals

An example of a pharmaceutical composition is a solution designed for parenteral administration. Although in many cases pharmaceutical solution formulations are provided in liquid form, appropriate for immediate use, such parenteral formulations may also be provided in frozen or in lyophilized form. The latter form is often used to enhance the stability of the active compound contained in the composition under a wider variety of storage conditions, as it is recognized by those skilled in the art that lyophilized preparations are generally more stable than their liquid counterparts. Such lyophilized preparations are reconstituted prior to use by the addition of one or more suitable pharmaceutically acceptable diluents such as sterile water for injection or sterile physiological saline solution.

In case of parenterals, they are prepared for storage as lyophilized formulations or aqueous solutions by mixing, as appropriate, the polypeptide having the desired degree of purity with one or more pharmaceutically acceptable carriers, excipients or stabilizers typically employed in the art (all of which are termed “excipients”), for example buffering agents, stabilizing agents, preservatives, isotonifiers, non-ionic detergents, antioxidants and/or other miscellaneous additives.

Buffering agents help to maintain the pH in the range which approximates physiological conditions. They are typically present at a concentration ranging from about 2 mM to about 50 mM Suitable buffering agents for use with the present invention include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffers (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additional possibilities are phosphate buffers, histidine buffers and trimethylamine salts such as Tris.

Preservatives are added to retard microbial growth, and are typically added in amounts of e.g. about 0.1%-2% (w/v). Suitable preservatives for use with the present invention include phenol, benzyl alcohol, meta-cresol, methyl paraben, propyl paraben, octade-cyldimethylbenzyl ammonium chloride, benzalkonium halides (e.g. benzalkonium chloride, bromide or iodide), hexamethonium chloride, alkyl parabens such as methyl or propyl paraben, catechol, resorcinol, cyclohexanol and 3-pentanol.

Isotonicifiers are added to ensure isotonicity of liquid compositions and include polyhydric sugar alcohols, preferably trihydric or higher sugar alcohols, such as glycerin, erythritol, arabitol, xylitol, sorbitol and mannitol. Polyhydric alcohols can be present in an amount between 0.1% and 25% by weight, typically 1% to 5%, taking into account the relative amounts of the other ingredients.

Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, omithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur-containing reducing agents, such as urea, glutathione, thioctic acid, sodium thioglycolate, thioglycerol, α-monothioglycerol and sodium thiosulfate; low molecular weight polypeptides (i.e. <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on the active protein weight.

Non-ionic surfactants or detergents (also known as “wetting agents”) may be present to help solubilize the therapeutic agent as well as to protect the therapeutic polypeptide against agitation-induced aggregation, which also permits the formulation to be exposed to shear surface stress without causing denaturation of the polypeptide. Suitable non-ionic surfactants include polysorbates (20, 80, etc.), polyoxamers (184, 188 etc.), Pluronic® polyols, polyoxyethylene sorbitan monoethers (Tween®-20, Tween®-80, etc.).

Additional miscellaneous excipients include bulking agents or fillers (e.g. starch), chelating agents (e.g. EDTA), antioxidants (e.g., ascorbic acid, methionine, vitamin E) and cosolvents.

The active ingredient may also be entrapped in microcapsules prepared, for example, by coascervation techniques or by interfacial polymerization, for example hydroxymethylcellulose, gelatin or poly-(methylmethacylate) microcapsules, in colloidal drug delivery systems (for example liposomes, albumin microspheres, microemulsions, nano-particles and nanocapsules) or in macroemulsions. Such techniques are disclosed in Remington's Pharmaceutical Sciences, supra.

Parenteral formulations to be used for in vivo administration must be sterile. This is readily accomplished, for example, by filtration through sterile filtration membranes.

Sustained Release Preparations

Suitable examples of sustained-release preparations include semi-permeable matrices of solid hydrophobic polymers containing the polypeptide or conjugate, the matrices having a suitable form such as a film or microcapsules. Examples of sustained-release matrices include polyesters, hydrogels (for example, poly(2-hydroxyethyl-methacrylate) or poly(vinylalcohol)), polylactides, copolymers of L-glutamic acid and ethyl-L-glutamate, non-degradable ethylene-vinyl acetate, degradable lactic acid-glycolic acid copolymers such as the ProLease® technology or Lupron Depot® (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for long periods such as up to or over 100 days, certain hydrogels release proteins for shorter time periods. When encapsulated polypeptides remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Pulmonary Delivery

Formulations suitable for use with a nebulizer, either jet or ultrasonic, will typically comprise the polypeptide or conjugate dissolved in water at a concentration of, e.g., about 0.01 to 25 mg of conjugate per mL of solution, preferably about 0.1 to 10 mg/mL. The formulation may also include a buffer and a simple sugar (e.g., for protein stabilization and regulation of osmotic pressure), and/or human serum albumin ranging in concentration from 0.1 to 10 mg/ml. Examples of buffers that may be used are sodium acetate, citrate and glycine. Preferably, the buffer will have a composition and molarity suitable to adjust the solution to a pH in the range of 3 to 9. Generally, buffer molarities of from 1 mM to 50 mM are suitable for this purpose. Examples of sugars which can be utilized are lactose, maltose, mannitol, sorbitol, trehalose, and xylose, usually in amounts ranging from 1% to 10% by weight of the formulation.

The nebulizer formulation may also contain a surfactant to reduce or prevent surface induced aggregation of the protein caused by atomization of the solution in forming the aerosol. Various conventional surfactants can be employed, such as polyoxyethylene fatty acid esters and alcohols, and polyoxyethylene sorbitan fatty acid esters. Amounts will generally range between 0.001% and 4% by weight of the formulation. An especially preferred surfactant for purposes of this invention is polyoxyethylene sorbitan monooleate.

Specific formulations and methods of generating suitable dispersions of liquid particles of the invention are described in WO 94/20069, U.S. Pat. Nos. 5,915,378, 5,960,792, 5,957,124, 5,934,272, 5,915,378, 5,855,564, 5,826,570 and 5,522,385, which are hereby incorporated by reference.

Formulations for use with a metered dose inhaler device will generally comprise a finely divided powder. This powder may be produced by lyophilizing and then milling a liquid conjugate formulation and may also contain a stabilizer such as human serum albumin (HSA). Typically, more than 0.5% (w/w) HSA is added. Additionally, one or more sugars or sugar alcohols may be added to the preparation if necessary. Examples include lactose maltose, mannitol, sorbitol, sorbitose, trehalose, xylitol, and xylose. The amount added to the formulation can range from about 0.01 to 200% (w/w), preferably from approximately 1 to 50%, of the conjugate present. Such formulations are then lyophilized and milled to the desired particle size.

The properly sized particles are then suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant. This mixture is then loaded into the delivery device.

Formulations for powder inhalers will comprise a finely divided dry powder containing conjugate and may also include a bulking agent, such as lactose, sorbitol, sucrose, or mannitol in amounts which facilitate dispersal of the powder from the device, e.g., 50% to 90% by weight of the formulation. The particles of the powder shall have aerodynamic properties in the lung corresponding to particles with a density of about 1 g/cm² having a median diameter less than 10 micrometers, preferably between 0.5 and 5 micrometers, most preferably of between 1.5 and 3.5 micrometers.

The powders for these devices may be generated and/or delivered by methods disclosed in U.S. Pat. Nos. 5,997,848, 5,993,783, 5,985,248, 5,976,574, 5,922,354, 5,785,049 and 5,654,007.

Mechanical devices designed for pulmonary delivery of therapeutic products, include but are not limited to nebulizers, metered dose inhalers, and powder inhalers, all of which are familiar to those of skill in the art. Specific examples of commercially available devices suitable for the practice of this invention are the Ultravent™ nebulizer, manufactured by Mallinckrodt, Inc., St. Louis, Mo.; the Acorn™ II nebulizer, manufactured by Marquest Medical Products, Englewood, Colo.; the Ventolin™ metered dose inhaler, manufactured by Glaxo Inc., Research Triangle Park, N.C.; the Spinhaler™ powder inhaler, manufactured by Fisons Corp., Bedford, Mass.; the “standing cloud” device of Inhale Therapeutic Systems, Inc., San Carlos, Calif.; the AIR TMinhaler manufactured by Alkermes, Cambridge, Mass.; and the AERxrm pulmonary drug delivery system manufactured by Aradigm Corporation, Hayward, Calif.

### EXAMPLES

Materials and Methods

Methods Used to Determine Amino Acids to be Modified

Accessible Surface Area (ASA)

The computer program Access (B. Lee and F. M. Richards, J. Mol.Biol. 55: 379-400 (1971)) version 2 (©1983 Yale University) are used to compute the accessible surface area (ASA) of the individual atoms in the structure. This method typically uses a probe-size of 1.4 Å and defines the Accessible Surface Area (ASA) as the area formed by the centre of the probe. Prior to this calculation all water molecules and all hydrogen atoms should be removed from the coordinate set, as should other atoms not directly related to the protein.

Fractional ASA of Side Chain

The fractional ASA of the side chain atoms is computed by division of the sum of the ASA of the atoms in the side chain by a value representing the ASA of the side chain atoms of that residue type in an extended ALA-x-ALA tripeptide. See Hubbard, Campbell & Thornton (1991) J.Mol.Biol. 220, 507-530. For this example the CA atom is regarded as a part of the side chain of glycine residues but not for the remaining residues. The following values are used as standard 100% ASA for the side chain:

| | | | | | |
|---|---|---|---|---|---|
| Ala | 69.23 | Å² | Leu | 140.76 | Å² |
| Arg | 200.35 | Å² | Lys | 162.50 | Å² |
| Asn | 106.25 | Å² | Met | 156.08 | Å² |
| Asp | 102.06 | Å² | Phe | 163.90 | Å² |
| Cys | 96.69 | Å² | Pro | 119.65 | Å² |
| Gln | 140.58 | Å² | Ser | 78.16 | Å² |
| Glu | 134.61 | Å² | Thr | 101.67 | Å² |
| Gly | 32.28 | Å² | Trp | 210.89 | Å² |
| His | 147.00 | Å² | Tyr | 176.61 | Å² |
| Ile | 137.91 | Å² | Val | 114.14 | Å² |

Residues not detected in the structure are defined as having 100% exposure as they are thought to reside in flexible regions.

Determining Distances Between Atoms

The distance between atoms is most easily determined using molecular graphics software, e.g. InsightII v. 98.0, MSI Inc.

General Considerations Regarding Amino Acid Residues to be Modified

As explained above, amino acid residues to be modified in accordance with the present invention are preferably those whose side chains are surface exposed, in particular those with more than about 25% of the side chain exposed at the surface of the molecule, and more preferably those with more than 50% side chain exposure. Another consideration is that residues located in receptor interfaces are preferably excluded so as to avoid or at least minimize possible interference with receptor binding or activation. A further consideration is that residues that are less than 10 Å from the nearest Lys (Glu, Asp) CB-CB (CA for Gly) should also be excluded. Finally, preferred positions for modification are in particular those that have a hydrophilic and/or charged residue, i.e. Asp, Asn, Glu, Gln, Arg, His, Tyr, Ser and Thr, positions that have an arginine residue being especially preferred.

Identifying G-CSF Amino Acid Residues for Modification

Taking G-CSF as an example, the information below illustrates the factors that generally should be taken into consideration when identifying amino acid residues to be modified in accordance with the present invention. Based on these considerations with respect to side chain exposure, residues located within and outside of the receptor interface, distance between atoms, and whether residues are charged and/or hydrophilic, it is possible to select amino acid residues that are suitable for modification in a given polypeptide to obtain a desired result.

Three-dimensional structures have been reported for human G-CSF by X-ray crystallography and by NMR spectroscopy, respectively (Proc. Natl. Acad. Sci. USA 90:5167-5171, 1993; Biochemistry 33:8453-8463, 1994). The X-ray structure of a complex between G-CSF and the BN-BC domains of the GCSFR receptor have been reported in Nature 401:713-717, 1999. Also, a 3D ensemble of 10 structures determined by NMR spectroscopy (Proc. Natl. Acad. Sci. USA 90:5167-5171, 1993) is available from the Protein Data Bank (PDB) (Bernstein et al., J. Mol. Biol. (1977) 112, pp. 535).

Aritomi et al., Nature 401:713-717, 1999 have described the X-ray structure of a complex between hG-CSF and the BN-BC domains of the G-CSF receptor. They identify the following hG-CSF residues as being part of the receptor binding interfaces: G4, P5, A6, S7, S8, L9, P10, Q11, S12, L15, K16, E19, Q20, L108, D109, D112, T115, T116, Q119, E122, E123, and L124. Thus, although it is possible to modify these residues, it is preferred that these residues are excluded from modification.

Using the 10 NMR structures of G-CSF identified above as input structures followed by a computation of the average ASA of the side chain, the following residues have been identified as having more than 25% ASA: M0, T1, P2, L3, G4, P5, A6, S7, S8, L9, P10, Q11, S12, F13, L14, L15, K16, C17, E19, Q20, V21, R22, K23, Q25, G26, D27, A29, A30, E33, K34, C36, A37, T38, Y39, K40, L41, H43, P44, E45, E46, V48, L49, L50, H52, S53, L54, I56, P57, P60, L61, S62, S63, P65, S66, Q67, A68, L69, Q70, L71, A72, G73, C74, S76, Q77, L78, S80, F83, Q86, G87, Q90, E93, G94, S96, P97, E98, L99, G100, P101, T102, D104, T105, Q107, L108, D109, A111, D112, F113, T115, T116, W118, Q119, Q120, M121, E122, E123, L124, M126, A127, P128, A129, L130, Q131, P132, T133, Q134, G135, A136, M137, P138, A139, A141, S142, A143, F144, Q145, R146, R147, S155, H156, Q158, S159, L161, E162, V163, S164, Y165, R166, V167, L168, R169, H170, L171, A172, Q173, P174.

Similarly, the following residues have more than 50% ASA: M0, T1, P2, L3, G4, P5, A6, S7, S8, L9, P10, Q11, S12, F13, L14, L15, K16, C17, E19, Q20, R22, K23, G26, D27, A30, E33, K34, T38, K40, L41, H43, P44, E45, E46, L49, L50, S53, P57, P60, L61, S62, S63, P65, S66, Q67, A68, L69, Q70, L71, A72, G73, S80, F83, Q90, G94, P97, E98, P101, D104, T105, L108, D112, F113, T115, T116, Q119, Q120, E122, E123, L124, M126, P128, A129, L130, Q131, P132, T133, Q134, G135, A136, A139, A141, S142, A143, F144, R147, S155, S159, E162, R166, V167, R169, H170, L171, A172, Q173, P174.

The molecular graphics program InsightII v.98.0 was used to determine residues having their CB atom (CA in the case of glycine) at a distance of more than 15 Å from the nearest amine group, defined as the NZ atoms of lysine and the N atom of the N-terminal residue T1. The following list includes the residues that fulfill this criteria in at least one of the 10 NMR structures. G4, P5, A6, S7, S8, L9, P10, Q11, L14, L15, L18, V21, R22, Q25, G26, D27, G28, A29, Q32, L35, C36, T38, Y39, C42, H43, P44, E45, E46, L47, V48, L49, L50, G51, H52, S53, L54, G55, I56, P57, W58, A59, P60, L61, S62, S63, C64, P65, S66, Q67, A68, L69, Q70, L71, A72, G73, C74, L75, S76, Q77, L78, H79, S80, G81, L82, F83, L84, Y85, Q86, G87, L88, L89, Q90, A91, L92, E93, G94, I95, S96, P97, E98, L99, G100, P101, T102, L103, D104, T105, L106, Q107, L108, D109, V110, A111, D112, F113, A114 T115, T116, I117, W118, Q119, Q120, M121, E122, E123, L124, G125, M126, A127, P128, A129, L130, Q131, P132, T133, Q134, G135, A136, M137, P138, A139, F140, A141, S142, A143, F144, Q145, R146, R147, A148, G149, G150, V151, L152, V153, A154, S155, H156, L157, Q158, S159, F160, L161, E162, V163, S164, Y165, R166, V167, L168, R169, H170, L171, A172, Q173, P174.

The InsightII v.98.0 program was similarly used to determine residues having their CB atom (CA atom in the case of glycine) at a distance of more than IOA from the nearest acidic group, defined as the CG atoms of aspartic acid, the CD atoms of glutamic acid and the C atom of the C-terminal residue P174. The following list includes the residues that fulfill this criteria in at least one of the 10 NMR structures. M0, T1, P2, L3, G4, P5, A6, S7, S8, L9, P10, Q11, S12, F13, L14, T38, Y39, K40, L41, C42, L50, G51, H52, S53, L54, G55, I56, P57, W58, A59, P60, L61, S62, S63, C64, P65, S66, Q67, A68, L69, Q70, L71, A72, G73, C74, L75, S76, Q77, L78, H79, S80, G81, L82, F83, L84, Y85, Q86, G87, L88, I117, M126, A127, P128, A129, L130, Q131, P132, T133, Q134, G135, A136, M137, P138, A139, F140, A141, S142, A143, F144, Q145, R146, R147, A148, G149, G150, V151, L152, V153, A154, S155, H156, L157, V167, L168, R169, H170, L171. By combining and comparing the above lists (or similar lists prepared for other polypeptides), it is possible to select individual amino acid residues for modification to result in a list containing a limited number of amino acid residues whose modification in a given polypeptide is likely to result in desired properties.

Methods for PEGylation of Single Chain G-CSF Dimer and Variants Thereof

PEGylation of Single Chain G-CSF Dimer and Variants Thereof in Solution

Single chain G-CSF dimer and variants thereof are PEGylated at a concentration of 250 μg/ml in 50 mM sodium phosphate, 100 mM NaCl, pH 8.5. The molar surplus of PEG is 100 times with respect to PEGylation sites on the protein. The reaction mixture is placed in a thermo mixer for 30 minutes at 37° C. at 1200 rpm. After 30 minutes, quenching of the reaction is obtained by adding a molar excess of glycine.

Cation exchange chromatography is applied to remove excess PEG, glycine and other by-products from the reaction mixture. The PEGylation reaction mixture is diluted with 20 mM sodium citrate pH 2.5 until the ionic strength is less than 7 mS/cm. pH is adjusted to 2.5 using 5 N HCl. The mixture is applied to an SP-sepharose FF column equilibrated with 20 mM sodium citrate pH 2.5. Unbound material is washed off the column using 4 column volumes of equilibration buffer. PEGylated protein is eluted in three column volumes by adding 20 mM sodium citrate, 750 mM sodium chloride. Pure PEGylated G-CSF is concentrated and buffer exchange is performed using VivaSpin concentration devices, molecular weight cut-off (mwco): 10 kDa.

PEGylation in Microtiter Plates of a Tagged Polypeptide with Single Chain G-CSF Dimer Activity

A polypeptide exhibiting single chain G-CSF dimer activity is expressed with a suitable tag, e.g. any of the tags exemplified in the general description above, and culture broth is transferred to one or more wells of a microtiter plate capable of immobilising the tagged polypeptide. When the tag is Met-Lys-His-Gln-His-Gln-His-Gln-His-Gln-His-Gln-His-Gln-Gln (SEQ ID NO:13), a nickel-nitrilotriacetic acid (Ni—NTA) HisSorb microtiter plate commercially available from QIAGEN can be used.

After immobilization of the tagged polypeptide to the microtiter plate, the wells are washed in a buffer suitable for binding and subsequent PEGylation followed by incubating the wells with the activated PEG of choice. As an example, M-SPA-PEG 5000 from Shear-water Corp. is used. The molar ratio of activated PEG to polypeptide should be optimized, but will typically be greater than 10:1, e.g. up to about 100:1 or higher. After a suitable reaction time at ambient temperature, typically around 1 hour, the reaction is stopped by removal of the activated PEG solution. The conjugated protein is eluted from the plate by incubation with a suitable buffer. Suitable elution buffers may contain imidazole, excess NTA or another chelating compound. The conjugated protein is assayed for biological activity and immunogenicity as appropriate. The tag may optionally be cleaved off using a method known in the art, e.g. using diaminopeptidase the Gln in pos −1 can be converted to pyroglutamyl with GCT (glutamylcyclotransferase) and finally cleaved off with PGAP (pyro-glutamyl-aminopeptidase), giving the untagged protein. The process involves several steps of metal chelate affinity chromatography. Alternatively, the tagged polypeptide may be conjugated.

Methods Used to Characterize Conjugated and Non-conjugated Single Chain G-CSF Dimer and Variants Thereof

Determination of the Molecular Size of Single Chain G-CSF Dimer and Variants Thereof

The molecular weight of conjugated or non-conjugated single chain G-CSF dimer or variants thereof is determined by either SDS-PAGE, gel filtration, matrix assisted laser desorption mass spectrometry or equilibrium centrifugation. As explained above, SDS-PAGE provides information on the “apparent molecular weight”. The actual molecular weight can advantageously be determined using mass spectrometry. SDS-PAGE is carried out using the NuPAGEO® kit (Novex high-performance pre-cast gels) from Invitrogen™. 15 μl of the samples are loaded onto NuPAGE® 4-12% Bis-Tris gels (Cat. No. NPO321) and eluted in NuPAGE® MES SDS running buffer (Cat. No. NPO002-02) for 35 minutes at 200 V and 120 mA.

Determination of Polypeptide Concentration

The concentration of a polypeptide can be measured using optical density measurements at 280 nm, an enzyme-linked immunoadsorption assay (ELISA), a radio-immunoassay (RIA), or other such immunodetection techniques well known in the art. Furthermore, the polypeptide concentration in a sample can be measured with the Biacore® instrument using a Biacore® chip coated with an antibody specific for the polypeptide.

Such an antibody can be covalently coupled to the Biacore® chip by various chemistries. Alternatively, the antibody can be non-covalently bound e.g. by means of an antibody specific for the Fc portion of the anti-polypeptide antibody. The Fc specific antibody is first coupled covalently to the chip. The anti-polypeptide antibody is then flowed over the chip and is bound by the first antibody in a directed fashion. Furthermore, biotinylated antibodies can be immobilised using a streptavidin coated surface (e.g. Biacore Sensor Chip SA®) (Real-Time Analysis of Biomolecular Interactions, Nagata and Handa (Eds.), 2000, Springer Verlag, Tokyo; Biacore 2000 Instrument Handbook, 1999, Biacore AB).

When the sample is flowed over the chip the polypeptide will bind to the coated antibody and the increase in mass can be measured. By using a preparation of the polypeptide in a known concentration, a standard curve can be established and subsequently the concentration of the polypeptide in the sample can be determined. After each injection of sample the sensor chip is regenerated by a suitable eluent (e.g. a low pH buffer) that removes the bound analyte.

Generally, the applied antibodies will be monoclonal antibodies raised against the wild-type polypeptide. Introduction of mutations or other manipulations of the wild-type polypeptide (extra glycosylations or polymer conjugations) may alter the recognition by such antibodies. Furthermore, such manipulations that give rise to an increased molecular weight of the polypeptide will result in an increased plasmon resonance signal. Consequently, it is necessary to establish a standard curve for every molecule to be tested.

Methods Used to Determine the in vitro and in vivo Activity of Conjugated and Non-conjugated Single Chain G-CSF Dimer and Variants Thereof

Primary Assay 1—in vitro Single Chain G-CSF Dimer Activity Assay

Proliferation of the murine cell line NFS-60 (obtained from Dr. J. Ihle, St. Jude Children's Research Hospital, Tennessee, USA) is dependent on the presence of active single chain G-CSF dimer in the growth medium. Thus, the in vitro biological activity of single chain G-CSF dimer and variants thereof can be determined by measuring the number of dividing NFS-60 cells after addition of a single chain G-CSF dimer sample to the growth medium followed by incubation over a fixed period of time.

NFS-60 cells are maintained in Iscoves DME Medium containing 10% w/w FBS (fetal bovine serum), 1% w/w Pen/Strep, 10 μg per liter hG-CSF and 2 mM Glutamax. Prior to sample addition, cells are washed twice in growth medium without hG-CSF and diluted to a concentration of 2.2×10⁵ cells per ml. 100 μl of the cell suspension is added to each well of a 96 well microtiter plate (Coming).

Samples containing conjugated or non-conjugated single chain G-CSF dimer or variants thereof are diluted to concentrations between 1.1×10⁻⁶ M and 1.1×10⁻¹³ M in the growth medium. 10 μl of each sample is added to 3 wells containing NFS-60 cells. A control consisting of 10 μl of mammalian growth medium is added to 8 wells on each microtiter plate. The cells are incubated for 48 hours (37° C., 5% CO₂) and the number of dividing cells in each well is quantified using the WST-1 cell proliferation agent (Roche Diagnostics GmbH, Mannheim, Germany). 0.01 ml WST-1 is added to the wells followed by incubation for 150 min. at 37° C. in a 5% CO₂ air atmosphere. The cleavage of the tetrazolium salt WST-1 by mitochondrial dehydrogenases in viable cells results in the formation of formazan that is quantified by measuring the absorbance at 450 nm. The number of viable cells in each well is hereby quantified.

Based on these measurements, dose-response curves for each conjugated and non-conjugated single chain G-CSF dimer molecule or variants thereof are calculated, after which the EC50 value for each molecule can be determined. This value is equal to the amount of active single chain G-CSF dimer protein that is necessary to obtain 50% of the maximum proliferation activity of non-conjugated human G-CSF. Thus, the EC50 value is a direct measurement of the in vitro activity of the given protein, a lower EC50 value indicating a higher specific activity.

Primary Assay 2—in vitro Single Chain G-CSF Dimer Activity Assay

The murine hematopoietic cell line BaF3 is transfected with a plasmid carrying the human G-CSF receptor and the promoter of the transcription regulator, fos, in front of the luciferase reporter gene. Upon stimulation of such a cell line with a single chain G-CSF dimer sample, a number of intracellular reactions lead to stimulation of fos expression, and consequently to expression of luciferase. This stimulation is monitored by the Steady-Glo™ Luciferase Assay System (Promega, Cat. No. E2510) whereby the in vitro activity of the G-CSF sample may be quantified.

BaF3/hGCSF-R/pfos-lux cells are maintained at 37° C. in a humidified 5% CO₂ atmosphere in complete culture media (RPMI-1640/HEPES (Gibco/BRL, Cat. No. 22400), 10% FBS (HyClone, characterized), 1×Penicillin/Streptomycin (Gibco/BRL, Cat. No. 15140-122), 1×L-Glutamine (Gibco/BRL, Cat. No. 25030-081), 10% WEHI-3 conditioned media (source of muIL-3), and grown to a density of 5×10⁵ cells/mL (confluent). The cells are reseeded at about 2×10⁴ cells/mL every 2-3 days.

One day prior to the assay, log-phase cells are resuspended at 2×10⁵ cells/mL in starving media (DMEM/F-12 (Gibco/BRL, Cat. No. 11039), 1% BSA (Sigma, Cat. No. A3675), 1×Penicillin/Streptomycin (Gibco/BRL, Cat. No. 15140-122), 1×L-Glutamine (Gibco/BRL, Cat. No. 25030-081), 0.1% WEHI-3 conditioned media) and starved for 20 hours. The cells are washed twice with Dulbecco's PBS (BioWhittaker Cat. No. 17512F), and tested for viability using Trypan Blue viability staining. The cells are resuspended in assay media (RPMI-1640 (phenol-red free, Gibco/BRL, Cat. No. 11835), 25 mM HEPES, 1% BSA (Sigma, Cat. No. A3675), 1×Penicillin/Streptomycin (Gibco/BRL, Cat. No. 15140-122), 1×L-Glutamine (Gibco/BRL, Cat. No. 25030-081) at 4×10⁶ cells/mL, and 50 μL are aliquotted into each well of a 96-well microtiter plate (Corning). Samples containing conjugated or non-conjugated single chain G-CSF dimer or variants thereof are diluted to concentrations between 1.1×10⁻⁷ M and 1.1×10⁻¹² M in the assay medium. 50 μl of each sample is added to 3 wells containing BaF3/hGCSF-R/pfos-lux cells. A negative control consisting of 50 μl of medium is added to 8 wells on each microtiter plate. The plates are mixed gently and incubated for 2 hours at 37° C. The luciferase activity is measured by following the Promega Steady-Glo™ protocol (Promega Steady-Glo™ Luciferase Assay System, Cat. No. E25 10). 100 μL of substrate is added per well followed by gentle mixing. Luminescence is measured on a TopCount luminometer (Packard) in SPC (single photon counting) mode.

Based on these measurements, dose-response curves for each conjugated and non-conjugated single chain G-CSF dimer molecule or variants thereof are calculated, after which the EC50 value for each molecule can be determined.

Secondary Assay—Binding Affinity of Single-chain G-CSF Dimer or Variants Thereof to the hG-CSF Receptor

Binding of single-chain G-CSF dimer or variants thereof to the hG-CSF receptor is studied using standard binding assays. The receptors may be purified extracellular receptor domains, receptors bound to purified cellular plasma membranes, or whole cells—the cellular sources being either cell lines that inherently express G-CSF receptors (e.g. NFS-60) or cells transfected with cDNAs encoding the receptors. The ability of single-chain G-CSF dimer or variants thereof to compete for the binding sites with native G-CSF is analyzed by incubating with a labeled G-CSF-analog, for instance biotinylated hG-CSF or radioiodinated hG-CSF. An example of such an assay is described by Yamasaki et al. (Drugs. Exptl. Clin. Res. 24:191-196 (1998)).

The extracellular domains of the hG-CSF receptor can optionally be coupled to Fc and immobilized in 96 well plates. Single-chain G-CSF dimer or variants thereof are subsequently added and the binding of these is detected using either specific anti-hG-CSF antibodies or biotinylated or radioiodinated hG-CSF.

Measurement of the in vivo Half-life of Conjugated and Non-conjugated Single-chain G-CSF Dimer and Variants Thereof

An important aspect of the invention is the prolonged biological half-life that is obtained by construction of a single-chain G-CSF dimer conjugated to a polymer moiety. The rapid decrease of hG-CSF serum concentrations has made it important to evaluate biological responses to treatment with conjugated and non-conjugated single-chain G-CSF dimers and variants thereof. Measurement of in vivo biological half-life can be carried out as described below.

Male Sprague Dawley rats (7 weeks old) are used. On the day of administration, the weights of the animals are measured (280-310 gram per animal). 100 μg per kg body weight of the non-conjugated and conjugated single-chain G-CSF dimer samples are each injected intravenously into the tail vein of five rats. At 1 minute, 30 minutes, 1, 2, 4, 6, 24 and 48 hours after the injection, 300 μl of blood is withdrawn from a tail vein of each rat. The blood samples are stored at room temperature for 1½ hours followed by isolation of serum by centrifugation (4° C., 5000×g for 20 minutes). The serum samples are stored at −80° C. until the day of analysis. The amount of active single-chain G-CSF dimer in the serum samples is quantified by a single-chain G-CSF dimer in vitro activity assay (see primary assay 2) after thawing the samples on ice.

Another example of an assay for the measurement of in vivo half-life of single-chain G-CSF dimer or variants thereof is described in U.S. Pat. No. 5,824,778, the content of which is hereby incorporated by reference.

Measurement of the in vivo Biological Activity in Healthy Rats of Conjugated and Non-conjugated Single-chain G-CSF Dimer and Variants Thereof

Measurement of the in vivo biological effects of single-chain G-CSF dimer in SPF Sprague Dawley rats (purchased from M & B A/S, Denmark) was used to evaluate the biological efficacy of conjugated and non-conjugated single-chain G-CSF dimer and variants thereof.

On the day of arrival the rats were randomly allocated into groups of 6. The animals were acclimatised for a period of 7 days wherein individuals in poor condition or at extreme weights were rejected. The weight range of the rats at the start of the acclimatization period was 250-270 g.

On the day of administration the rats were fasted for 16 hours followed by subcutaneous injection of 100 μg per kg body weight of Neupogen® or conjugated or non-conjugated single-chain G-CSF dimer or a variant thereof. Each single-chain G-CSF dimer sample was injected into a group of 6 randomized rats. Blood samples of 300 μl EDTA stabilised blood were drawn from a tail vein of the rats prior to dosing and at 6, 12, 24, 36, 48, 72, 96, 120, 144 and 168 hours after dosing. The blood samples were analyzed for total white blood cell counts. On the basis of these measurements the biological efficacy of conjugated and non-conjugated single-chain G-CSF dimer and variants thereof was evaluated.

Further examples of assays that can be used to measure the in vivo biological activity of single-chain G-CSF dimer or variants thereof are described in U.S. Pat. Nos. 5,681,720, 5,795,968, 5,824,778, 5,985,265 and by Bowen et al., Experimental Hematology 27:425-432 (1999).

Measurement of the in vivo Biological Activity in Rats with Chemotherapy-induced Neutropenia of Conjugated and Non-conjugated Single-chain G-CSF Dimer and Variants Thereof

SPF Sprague Dawley rats were purchased from M & B A/S, Denmark. On the day of arrival the rats were randomly allocated into groups of 6. The animals were acclimatised for a period of 7 days wherein individuals in poor condition or at extreme weights were rejected. The weight range of the rats at the start of the acclimatization period was 250-270 g.

24 hours before administration of the single-chain G-CSF dimer samples the rats were injected i.p. with 50-100 mg per kg body weight of cyclophosphamide (CPA). At day 0, 100 μg per kg body weight of single-chain G-CSF dimer or a variant thereof was injected s.c. Each single-chain G-CSF dimer sample was injected into a group of 6 randomized rats. In addition, 5 μg per kg body weight of Neupogen® was injected s.c. into a group of 6 randomized rats at time 0, 24, 48, 72 and 96 hours. Blood samples of 300 μl EDTA stabilized blood were drawn from a tail vein of the rats prior to dosing and at 6, 12, 24, 36, 48, 72, 96, 120, 144 and 168 hours after dosing. The blood samples were analyzed for total white blood cell counts. On the basis of these measurements the biological efficacy of conjugated and non-conjugated single-chain G-CSF dimer and variants thereof was evaluated.

Determination of Polypeptide-receptor Binding Affinity (on- and off-rate)

The strength of the binding between a receptor and ligand can be measured using an enzyme-linked immunoadsorption assay (ELISA), a radio-immunoassay (RIA), or other such immunodetection techniques well known in the art. The ligand-receptor binding interaction may also be measured with the Biacore® instrument (Zhou et al., Biochemistry, 1993, 32, 8193-98; Faegerstram and O'Shannessy, 1993, In Handbook of Affinity Chromatography, 229-52, Marcel Dekker, Inc., NY).

The Biacore® technology allows one to bind receptor to a gold surface and to flow ligand over it. Plasmon resonance detection gives direct quantification of the amount of mass bound to the surface in real time. This technique yields both on and off-rate constants and thus a ligand-receptor dissociation constant and an affinity constant can be directly determined.

In vitro Immunogenicity test of Single-chain G-CSF Dimer Conjugates

The reduced immunogenicity of a conjugate of the invention can be determined by use of an ELISA method measuring the immunoreactivity of the conjugate relative to a reference molecule or preparation. The reference molecule or preparation is normally a recombinant human G-CSF preparation such as Neupogen® or another recombinant human G-CSF preparation, e.g. an N-terminally PEGylated rhG-CSF molecule as described in U.S. Pat. No. 5,824,784. The ELISA method is based on antibodies from patients treated with one of these recombinant G-CSF preparations. The immunogenicity is considered to be reduced when the conjugate of the invention has a statistically significant lower response in the assay than the reference molecule or preparation.

Neutralisation of Activity in the Single-chain G-CSF Dimer Activity Assay

The neutralisation of single-chain G-CSF dimer conjugates by anti-G-CSF sera is analysed using the primary single-chain G-CSF dimer assay 2 described above.

Sera from patients treated with the G-CSF reference molecule or from immunised animals are used. Sera are added either in a fixed concentration (dilution 1:20-1:500 (pt sera) or 20-1000 ng/ml (animal sera)) or in five-fold serial dilutions of sera starting at 1:20 (pt sera) or 1000 ng/ml (animal sera). Single-chain G-CSF dimer conjugate is added either in seven fold-dilutions starting at 10 nM or in a fixed concentration (1-100 pM) in a total volume of 80 μl DMEM (Dulbecco's modified Eagle's medium)+10% FCS (fetal calf serum). The sera are incubated for 1 hr. at 37° C. with single-chain G-CSF dimer conjugate.

The samples (0.01 ml) are then transferred to 96 well tissue culture plates containing NFS-60 cells in 0.1 ml DMEM media. The cultures are incubated for 48 hours at 37° C. in a 5% CO₂ air atmosphere. 0.01 ml WST-1 (WST-1 cell proliferation agent, Roche Diagnostics GmbH, Mannheim, Germany) is added to the cultures and incubated for 150 min. at 37° C. in a 5% CO₂ air atmosphere. The cleavage of the tetrazolium salt WST-1 by mitochondrial dehydrogenases in viable cells results in the formation of formazan that is quantified by measuring the absorbance at 450 nm.

When single-chain G-CSF dimer conjugate samples are titrated in the presence of a fixed amount of serum, the neutralising effect is defined as fold inhibition (FI) quantified as EC50(with serum)/EC50 (without serum). The reduction of antibody neutralisation of G-CSF variant proteins is defined as

$$( 1 - \frac{( FI variant - 1 )}{( FI wt - 1 )} ) \times 100 \%$$

### Example 1

Construction and Cloning of a Synthetic Gene Encoding Single-chain G-CSF Dimer

A DNA fragment, encoding the YAP3 signal sequence (WO 98/32867, SEQ ID NO:2), the TA57 leader sequence (WO 98/32867, SEQ ID NO:3), a KEX2 protease recognition site (AAAAGA), G-CSF copy 1 (SEQ ID NO:4) and G-CSF copy 2 (SEQ ID NO:5) in the single-chain G-CSF dimer was synthesised following the general procedure described by Stemmer et al. (1995), Gene 164, pp. 49-53. A Bam HI and an Xba I digestion site were introduced upstream and downstream, respectively, of the gene. The DNA fragment was cloned into the Bam HI and Xba I digestion sites in plasmid pJSO37 (Okkels, Ann. New York Acad. Sci. 782:202-207, 1996) using standard DNA techniques, resulting in plasmid pscG-CSF.

Another DNA fragment, consisting of a Bam HI digestion site, the Kozak consensus sequence (Kozak, M., J Mol Biol, August 1987; 196(4):947-50), a sequence encoding the hG-CSF signal peptide (SEQ ID NO:7), G-CSF copy 1 with the codon usage optimised for expression in CHO cells (SEQ ID NO:8) and G-CSF copy 2 (SEQ ID NO:5) in the single-chain G-CSF dimer and an Xba I digestion site, was also synthesised following the general procedure described by Stemmer et al. (1995), Gene 164, pp. 49-53. The DNA fragment was inserted into the Bam HI and Xha I digestion sites in plasmid pcDNA3.1 (+) (Invitrogen) using standard DNA techniques. This resulted in plasmid pscG-CSFCHO.

### Example 2

Expression of Single-chain G-CSF Dimer in Saccharomyces cerevisiae

Expression of the single-chain G-CSF dimer in S. cerevisiae YNG318 (available from the American Type Culture Collection, VA, USA as ATCC 208973) was performed using the following procedure: 10 μl 0.2 μg/μl pscG-CSF, 10 μl salmon testes carrier DNA and 100 μl competent S. cerevisiae YNG318 cells were mixed and 600 μl 25% PEG 4000 containing 0.1 M lithium acetate was added. The cells were incubated at 37° C for 30 minutes and placed in a 42° C. water bath for 15 minutes. The cells were pelleted by centrifugation (4000 rpm, 2 minutes), the supernatant was discharged and the cells were resuspended in non-selective YPD medium (1% w/w yeast extract (Difco), 2% w/w peptone bacto (Difco), 3% w/w dextrose (Roquette)). Following incubation at 37° C. for 1 hour, the cells were plated on selective SC-without-uracil medium (7.5 g per liter yeast nitrogen base w/o amino acids (Difco), 11.3 g per liter Bernstein acid (Merck), 6.8 g per liter NaOH (Merck), 5.6 g per liter casamino acid w/o vitamin, 0.1 g per liter tryptophan, 20 g per liter glucose (Sigma), 0.1 g per liter threonine).

One transformant of S. cerevisiae containing plasmid pscG-CSF with the ability to grow on selective medium without uracil was innoculated in 1000 ml liquid SC-without-uracil medium containing 0.2% Tween 80 at 37° C. for 72 hours. The yeast culture was centrifuged (4000 rpm, 5 minutes) and the supernatant was isolated. Expression of single-chain G-CSF dimer was verified by Western Blot analysis using the ImmunoPure® Ultra-Sensitive ABC Rabbit IgG Staining kit (Pierce) and a polyclonal antibody against hG-CSF (Pepro Tech EC Ltd.). It was observed that the protein had the correct size.

### Example 3

Expression of Single-chain G-CSF Dimer in Chinese Hamster Ovary (CHO) Cells

The day before transfection the CHO K1 cell line (ATCC #CC1-61) was seeded in a T-25 flask in 5 ml DMEM/F-12 medium (Gibco # 31330-038) supplemented with 10% FBS and penicillin/streptomycin. The following day (at nearly 100% confluency) the transfection was prepared: 90 μl DMEM medium without supplements was aliquoted into a 14 ml polypropylene tube (Coming). 10 μl Fugene 6 (Roche) was added directly into the medium and incubated for 5 min at room temperature. In the meantime 5 μg plasmid pscG-CSFCHO was aliquoted into another 14 ml polypropylene tube. After incubation the Fugene 6 mix was added directly to the DNA solution and incubated for 15 min at room temperature. After incubation the whole volume was added drop-wise to the cell medium.

The next day the medium was exchanged with fresh medium containing 360 μg/ml hygromycin (Gibco). Every day hereafter the selection medium was renewed until the primary transfection pool had reached 100% confluency. The primary transfection pool was subsequently sub-cloned by limited dilution (300 cells seeded in five 96-well plates) whereby stable cell lines expressing single chain G-CSF dimer was obtained. One stable cell line expressing 8 mg/L single chain G-CSF dimer as determined by ELISA (Quantikine Human G-CSF Immunoassay, R&D Systems Cat. No. DCS50) was transferred to T-175 flasks.

Cells from 1-2 confluent T-175 flasks were transferred to one roller bottle (1700 cm²) in 300 ml DMEM/F-12 medium (Life Technologies # 31330) supplemented with 10% FBS and penicillin/streptomycin (P/S). The medium was exchanged every second day until the bottle was nearly confluent. The medium was then changed to 300 ml serum-free Ultra-CHO medium (BioWhittaker # 12-724) supplemented with 1/1000 EX-CYTE (Serologicals Proteins # 81129N) and P/S. After 4 days (where the medium was exchanged every second day) the roller bottle was ready for production and the medium was shifted to the production medium: DMEM/F-12 medium without phenol red (Life Technologies # 21041) supplemented with 1/100 ITSA (Life Technologies # 51300-044) [ITSA: Insulin (1.0 g/L)—Transferrin (0.55 g/L)—Selenium (0.67 mg/L) supplement for Adherent cultures], 1/1000 EC-CYTE and P/S. During the production period the medium was exchanged every day.

### Example 4

Purification of Single-chain G-CSF Dimer from Yeast Culture Supernatants

1000 ml of supernatant from a cultivation of a S. cerevisiae strain expressing single-chain G-CSF dimer was sterile filtered. The ionic strength in the culture supernatant was measured. To lower the ionic strength before application onto a cation-exchange column, the supernatant was diluted with 10 mM Na acetate pH 4.5 to a volume of 5000 ml. Finally, the pH was adjusted to pH 4.5.

The diluted sample was applied to an SP Sepharose FF column (20 ml) equilibrated in 50 mM Na acetate, pH 4.5 operated at a flow rate of 5 ml/min. Following application, the column was washed with equilibration buffer until the A₂₈₀ in the column effluent reached a stable level. Elution was performed at a flow rate of 3 ml/min using a linear gradient of NaCl (0 M to 0.75 M) in 50 mM Na acetate, pH 4.5 over 20 column volumes. Fractions of 2.4 ml were collected. The fractions containing single-chain G-CSF dimer were pooled. Sample preparation before performing hydrophobic interaction chromatography was made by adding 4 volumes of 1 M ammonium sulphate, pH 8 to reach a final concentration of 800 mM ammonium sulphate. The pH of the sample was measured before loading and was found to be 8.

The sample was loaded onto a column packed with 5 ml ToyoPearl Phenylsepharose-650 equilibrated with 10 column volumes of 1 M ammonium sulphate, pH 8 at a flow rate of 2 ml/min. The column was washed with 0.8 M ammonium sulphate until the A₂₈₀ in the column effluent reached a stable level. Elution of the column was performed using a linear gradient (0.8 M ammonium sulphate, pH 8, to MilliQ filtered water) for 60 column volumes at a flow rate of 3 ml/min. Fractions of 2.5 ml were collected.

The concentration of single-chain G-CSF dimer in the fractions from the HIC column was determined by ELISA (Quantikine G-CSF ELISA assay, R&D Systems catalogue number DCS50).

The amino acid sequence of the single-chain G-CSF dimer is given in SEQ ID NO:6.

### Example 5

Purification of Single-chain G-CSF Dimer from CHO Cell Culture Supernatants

Culture medium from the roller bottle cultivations of CHO cells expressing single chain G-CSF dimer was harvested and sterile filtered using a 0.22 μm filter. The conductivity was adjusted to 75 mS/cm using 4 M sodium chloride and the conditioned medium was applied on a column of Phenyl Toyo Pearl 650 S resin equilibrated with 20 mM sodium phosphate, pH 7.0; 750 mM sodium chloride. The column was washed to stable base line using the equilibration buffer and eluted stepwise using 20 mM sodium phosphate, pH 7.0. Fractions containing sc-GCSF were pooled and adjusted with 50 mM sodium acetate to reach pH 4.5 and a conductivity below 7 mS/cm. The adjusted fractions were applied on a SP-sepharose FF (Pharmacia) at a flow rate of 150-300 cm/hour. The column was washed and eluted by a step gradient 0-100% B-buffer (750 mM sodium chloride, 50 mM sodium acetate (pH 4.5) with increments of 10% B-buffer at a flow rate of 150 cm/hour. The fractions containing single chain G-CSF dimer were subsequently pooled.

The amino acid sequence of the single-chain G-CSF dimer is given in SEQ ID NO:6.

### Example 6

Characterization of Purified Single-chain G-CSF Dimer from CHO Cells

Purity

The purity of purified single-chain G-CSF dimer was determined by reverse phase HPLC. A single-chain G-CSF dimer sample was applied to a Vydac C,₈ reverse phase column (0.21×5 cm) and isocratically eluted with buffer A (0.1% TFA in water) and buffer B (0.1% TFA in acetonitril) using the following gradient: 0-5 minutes: 100% buffer A, 5-15 minutes: 0-40% buffer B, 15-35 minutes: 40-100% buffer B, 35-40 minutes; 100-0% buffer B. The purity was determined to be higher than 90%. The RP-HPLC result was confirmed by SDS-PAGE analysis.

Identity

The identity of purified single-chain G-CSF dimer was confirmed by N-terminal amino acid sequencing.

Size

The purified single-chain G-CSF dimer was analyzed by SDS-PAGE. A single band having an apparent molecular weight of 38 kDa was detected. MALDI-TOF mass spectrometry of purified single-chain G-CSF dimer showed that the size of the protein was 38.8 kDa corresponding to 2 times the molecular weight of hG-CSF.

Concentration

An estimate of the single chain G-CSF dimer concentration in purified samples was obtained by spectrophotometric methods. By measuring the absorbance at 280 nm and using a theoretical extinction coefficient of 0.83, the protein concentration was calculated. A more accurate protein determination was obtained by amino acid analysis. Amino acid analysis performed on purified single chain G-CSF dimer revealed that the experimentally determined amino acid composition was in agreement with the expected amino acid composition based on the DNA sequence.

### Example 7

Construction of Single-chain G-CSF Dimer Variants

Specific substitutions of existing amino acids in the single chain G-CSF dimer to other amino acid residues, e.g. the specific substitutions discussed above in the general description, were introduced using standard DNA techniques known in the art. The new single chain G-CSF dimer variants were made using plasmid pscG-CSFCHO as DNA template in the PCR reactions. The following variants were constructed: Single chain G-CSF dimer (K16R)_{copy} 1,2 (i.e. with the substitution K16R in copy 1 and copy 2), single chain G-CSF dimer (K16R K34R)_{copy} 1,2, and single chain G-CSF dimer (K16R K34R K40R)_{copy 1,2}. The variants were expressed in CHO cells and purified as described in Example 5.

### Example 8

Covalent Attachment of SPA-PEG to hG-CSF or Variants Thereof

SPA-PEG 5000, SPA-PEG 12000 and SPA-PEG 20000 (Shearwater Corp.) were covalently linked to single chain G-CSF dimer and variants thereof purified from CHO cells as described above (“PEGylation of single chain G-CSF dimer and variants thereof in solution”). The apparent sizes of the PEGylated compounds as determined by SDS-PAGE are listed below.

| | Apparent |
|---|---|
| Molecule | size on SDS-PAGE (kDa) |
| Single-chain G-CSF dimer | 38 |
| Single-chain G-CSF dimer PEG5000 | 80-120 |
| Single-chain G-CSF dimer PEG12000 | 120-180 |
| Single-chain G-CSF dimer PEG20000 | 120-200 |

| | Apparent size on |
|---|---|
| Molecule | SDS-PAGE (kDa) |
| Single-chain G-CSF dimer (K16R)_{copy 1,2} | 38 |
| Single-chain G-CSF dimer (K16R)_{copy 1,2} PEG5000 | 60-110 |
| Single-chain G-CSF dimer (K16R)_{copy 1,2} PEG12000 | 120-160 |
| Single-chain G-CSF dimer (K16R K34R)_{copy 1,2} | 38 |
| Single-chain G-CSF dimer (K16R K34R)_{copy 1,2} | 55-110 |
| PEG5000 | |
| Single-chain G-CSF dimer (K16R K34R)_{copy 1,2} | 120-160 |
| PEG12000 | |
| Single-chain G-CSF dimer (K16R K34R K40R)_{copy 1,2} | 38 |
| Single-chain G-CSF dimer (K16R K34R K40R)_{copy 1,2} | 55-110 |
| PEG5000 | |
| Single-chain G-CSF dimer (K16R K34R K40R)_{copy 1,2} | 120-160 |
| PEG12000 | |

### Example 9

In vitro Biological Activity of Non-conjugated and Conjugated hG-CSF and Variants Thereof

The in vitro biological activities of conjugated and non-conjugated single chain G-CSF dimer and variants thereof were measured as described above in “Primary assay 2—in vitro single chain G-CSF dimer activity assay”. The in vitro bioactivities, determined on the basis of the measured EC50 values for each variant with and without conjugation of SPA-PEG to the available PEGylation sites, are listed below. The values have been normalized with respect to the EC50 value of non-conjugated hG-CSF (Neupogen®), which in this assay is 30 pM. This value was measured simultaneously with that of the variants under identical assay conditions. The values in the table thus indicate % specific activity relative to the activity of non-conjugated hG-CSF.

| | Specific activity |
|---|---|
| | (% of |
| Molecule | Neupogen ®) |
| Single-chain G-CSF dimer | 214 |
| Single-chain G-CSF dimer PEG5000 | 120 |
| Single-chain G-CSF dimer PEG12000 | 62 |
| Single-chain G-CSF dimer PEG20000 | 25 |
| Single-chain G-CSF dimer (K16R)_{copy 1,2} | 135 |
| Single-chain G-CSF dimer (K16R K34R)_{copy 1,2} | 329 |
| Single-chain G-CSF dimer (K16R K34R)_{copy 1,2} | 19 |
| PEG5000 | |
| Single-chain G-CSF dimer (K16R K34R)_{copy 1,2} | 8 |
| PEG12000 | |
| Single-chain G-CSF dimer (K16R K34R K40R)_{copy 1,2} | 300 |
| Single-chain G-CSF dimer (K16R K34R K40R)_{copy 1,2} | 10 |
| PEG5000 | |
| Single-chain G-CSF dimer (K16R K34R K40R)_{copy 1,2} | 10 |
| PEG12000 | |

The above results show that the non-PEGylated single-chain dimers all have a higher in vitro activity than that of hG-CSF. This increased activity is believed to be due to an increased binding affinity. It may also be seen that PEGylation substantially reduces the activity, and that this reduction in activity is greater when the MW of the PEG molecules is higher.

### Example 10

In vivo Biological Activity in Healthy Rats of Non-conjugated and Conjugated Single Chain hG-CSF Dimer and Variants Thereof

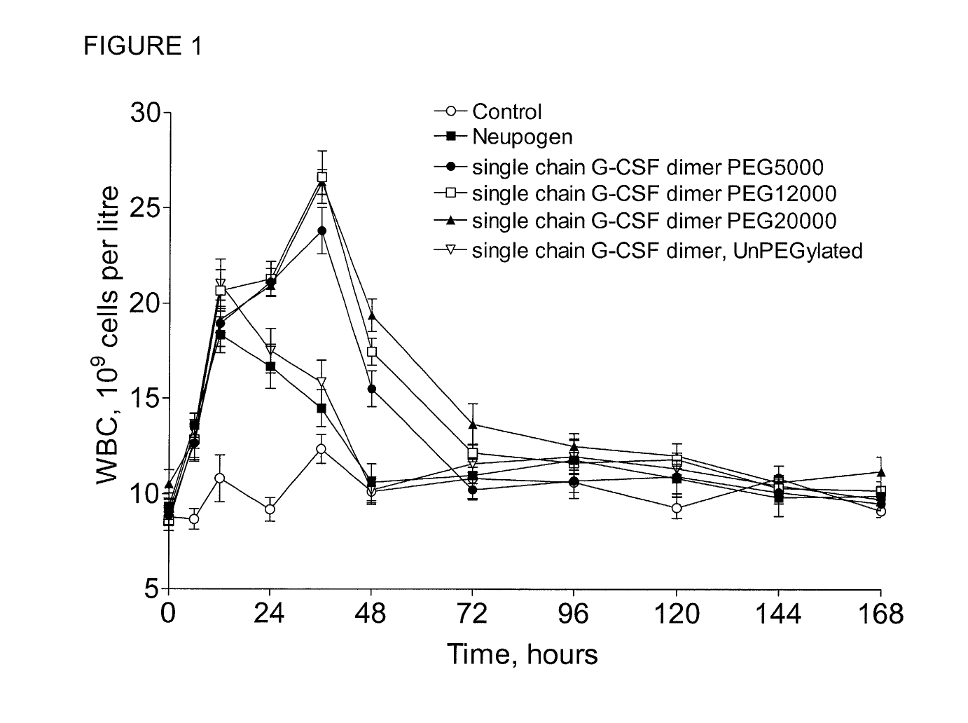

The in vivo biological activities of non-conjugated hG-CSF (Neupogen®), non-conjugated single-chain G-CSF dimer, SPA-PEG 5000 conjugated single-chain G-CSF dimer, SPA-PEG 12000 conjugated single-chain G-CSF dimer, and SPA-PEG 20000 conjugated single-chain G-CSF dimer in healthy rats were measured as described above (“Measurement of the in vivo biological activity in healthy rats of conjugated and non-conjugated single-chain G-CSF dimer and variants thereof”). The results are shown in FIG. 1.

The initial formation of white blood cells (first 12 hours) in rats receiving any of the four preparations of single-chain G-CSF dimer occurred with the same rate as observed in rats receiving hG-CSF (Neupogen®). Thus, single-chain G-CSF dimer stimulates formation of white blood cells in vivo as efficiently as hG-CSF. The level of white blood cells in rats receiving either hG-CSF (Neupogen®) or non-conjugated single-chain G-CSF dimer returned to normal 48 hours after injection. In rats receiving SPA-PEG 5000 conjugated single-chain G-CSF dimer, SPA-PEG 12000 conjugated single-chain G-CSF dimer, and SPA-PEG 20000 conjugated single-chain G-CSF dimer, the level of white blood cells returned to normal only after about 72 hours, 72 hours and 96 hours, respectively, after injection. Thus, the duration of action of the PEGylated single-chain G-CSF dimers is significantly longer than that of hG-CSF (Neupogen®).

### Example 11

In vivo Biological Activity in Rats with Chemotherapy-induced Neutropenia of Non-conjugated and Conjugated Single-chain G-CSF Dimer and Variants Thereof

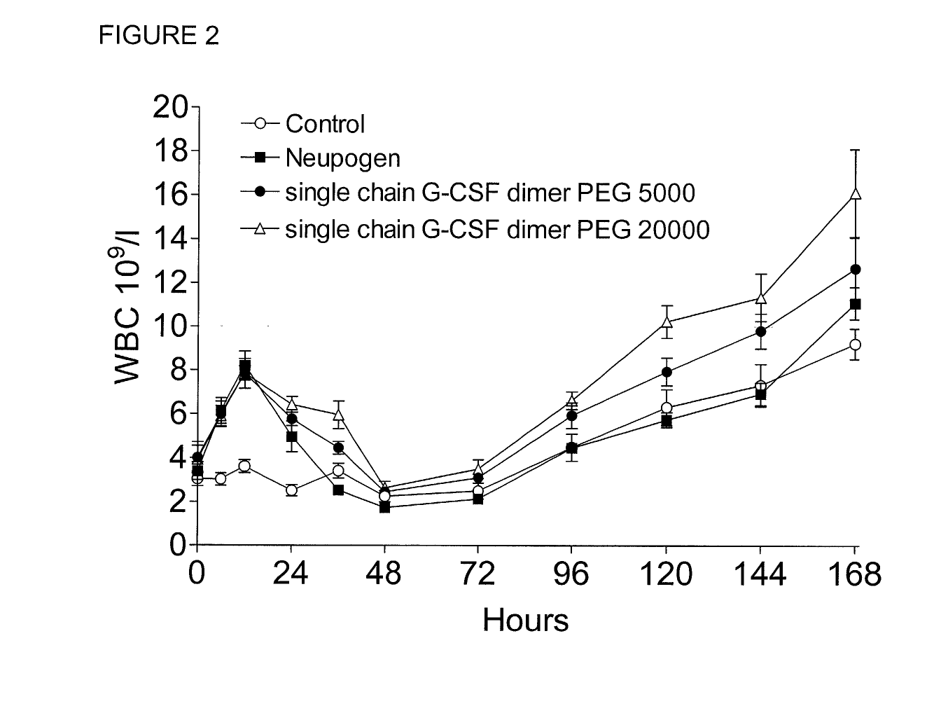

The in vivo biological activities of non-conjugated hG-CSF (Neupogen®), SPA-PEG5000 conjugated single-chain G-CSF dimer, and SPA-PEG20000 conjugated single-chain G-CSF dimer in rats with chemotherapy-induced neutropenia were measured as described above (“Measurement of the in vivo biological activity in rats with chemotherapy-induced neutropenia of conjugated and non-conjugated single-chain G-CSF dimer and variants thereof”) using 50 mg per kg body weight of cyclophosphamide (CPA). The results are shown in FIG. 2.

The initial stimulation of white blood cell formation during the first 12 hours was identical in rats receiving hG-CSF (Neupogen®), SPA-PEG5000 conjugated single-chain G-CSF dimer, or SPA-PEG20000 conjugated single-chain G-CSF dimer. After 36 hours the number of white blood cells (WBC) in the Neupogen®-treated rats dropped to the level that was observed in the untreated group (about 2×10⁹ cells per liter). At this point the rats were neutropenic. The level of white blood cells in the Neupogen-treated group reached normal levels (10×10⁹ cells per liter) after 168 hours.

The level of white blood cells in the two groups treated with SPA-PEG 5000 conjugated single-chain G-CSF dimer and SPA-PEG 20000 conjugated single-chain G-CSF dimer dropped to a minimum of about 2×19 cells per liter only after 48 hours. The white blood cell levels in the two groups were back to normal after 144 hours and 120 hours, respectively. Thus, the two PEG-conjugated single chain G-CSF dimer compounds were able to both relieve the degree of neutropenia and to reduce the time until the white blood cell levels were back to normal (the duration of neutropenia) significantly from 132 hours in the Neupogen®-treated group to 96 hours and 72 hours, respectively, in the groups treated with either SPA-PEG 5000 conjugated single chain G-CSF dimer or SPA-PEG 20000 conjugated single chain G-CSF dimer.

While the foregoing invention has been described in some detail for purposes of clarity and understanding, it will be clear to one skilled in the art from a reading of this disclosure that various changes in form and detail can be made without departing from the true scope of the invention. It is understood that the examples and embodiments described herein are for illustrative purposes only and that various modifications or changes in light thereof will be suggested to persons skilled in the art and are to be included within the spirit and purview of this application and scope of the appended claims. For example, all the techniques and apparatus described above may be used in various combinations. All publications, patents, patent applications, and/or other documents cited in this application are incorporated herein by reference in their entirety for all purposes to the same extent as if each individual publication, patent, patent application, and/or other document were individually indicated to be incorporated herein by reference in its entirety for all purposes.

We claim:

1. A single-chain multimeric polypeptide having G-CSF activity, comprising at least two monomeric units independently selected from (a) hG-CSF with the amino acid sequence shown in SEQ ID NO:1 and (b) variants of hG-CSF, said monomeric units being linked via a peptide bond or a peptide linker, the polypeptide comprising at least one covalently bound polymer molecule selected from the group consisting of linear and branched polyalkylene oxides.

2. The single-chain multimeric polypeptide of claim 1, wherein the polymer molecule is polyethylene glycol.

3. The single-chain multimeric polypeptide of claim 2, wherein at least one polyethylene glycol molecule is bound to a lysine residue.

4. The single-chain multimeric polypeptide of claim 1, wherein at least one of said monomeric units has the amino acid sequence of hG-CSF (SEQ ID NO:1).

5. The single-chain multimeric polypeptide of claim 4, comprising two monomeric units, wherein both of said monomeric units have the amino acid sequence of hG-CSF (SEQ ID NO:1).

6. The single-chain multimeric polypeptide of claim 1, wherein at least one of said monomeric units is a variant of hG-CSF comprising at least one substitution, addition or deletion compared to SEQ ID NO:1.

7. The single-chain multimeric polypeptide of claim 6, wherein at least one of said monomeric units is a variant of hG-CSF comprising at least one amino acid residue modification selected from the group consisting of: introduction of a lysine, cysteine, aspartic acid, glutamic acid or histidine residue; and removal of a lysine, cysteine, aspartic acid, glutamic acid or histidine residue.

8. The single-chain multimeric polypeptide of claim 6, comprising two monomeric units, wherein both of said monomeric units have at least one amino acid residue modification compared to hG-CSF.

9. The single-chain multimeric polypeptide of claim 1, comprising two monomeric units, wherein one of said monomeric units has the amino acid sequence of hG-CSF (SEQ ID NO:1) and the other monomeric unit is a variant of hG-CSF

10. A single-chain multimeric polypeptide having G-CSF activity, comprising at least two monomeric units independently selected from (a) hG-CSF with the amino acid sequence shown in SEQ ID NO:1 and (b) variants of hG-CSF, said monomeric units being linked via a peptide bond or a peptide linker, wherein the polypeptide has at least one non-polypeptide moiety covalently bound to an attachment group of the polypeptide and exhibits an in vitro bioactivity in the range of about 2-30% of the bioactivity of non-conjugated hG-CSF as determined by the luciferase assay described herein.

11. The single-chain multimeric polypeptide of claim 10, comprising two monomeric units.

12. The single-chain multimeric polypeptide of claim 11, wherein each of said two monomeric units comprises the amino acid sequence of hG-CSF as shown in SEQ ID NO:1.

13. The single-chain multimeric polypeptide of claim 10, wherein at least one monomeric unit is a variant of hG-CSF with at least one amino acid residue modification selected from the group consisting of: introduction of a lysine, cysteine, aspartic acid, glutamic acid or histidine residue; removal of a lysine, cysteine, aspartic acid, glutamic acid or histidine residue; introduction of an N- or O-glycosylation site; and removal of an N- or O-glycosylation site.

14. The single-chain multimeric polypeptide of claim 10, wherein at least one monomeric unit differs from the corresponding wild-type polypeptide in that least one attachment site for a non-polypeptide moiety has been introduced in a position that in the wild-type polypeptide is occupied by a surface-exposed amino acid residue, and/or wherein at least one amino acid residue that is surface-exposed in the wild-type polypeptide has been removed.

15. The single-chain multimeric polypeptide of claim 10, comprising at least one covalently bound non-polypeptide moiety selected from the group consisting of polymer molecules, lipophilic compounds, oligosaccharide moieties and organic derivatizing agents.

16. The single-chain multimeric polypeptide of claim 15, comprising at least one covalently bound polymer molecule selected from the group consisting of linear and branched polyalkylene oxides.

17. The single-chain multimeric polypeptide of claim 16, wherein the polymer molecule is polyethylene glycol.

18. The single-chain multimeric polypeptide of claim 11, wherein at least one of said two monomers differs from wild-type human G-CSF in that at least one lysine residue has been removed.

19. The single-chain multimeric polypeptide of claim 11, wherein at least one of said two monomers differs from wild-type human G-CSF in that at least one lysine residue has been introduced.

20. The single-chain multimeric polypeptide conjugate of claim 10 having an in vitro bioactivity in the range of about 3-25% of the bioactivity of non-conjugated hG-CSF as determined by the luciferase assay described herein.

21. The single-chain multimeric polypeptide conjugate of claim 10 having an in vitro bioactivity in the range of about 4-20% of the bioactivity of non-conjugated hG-CSF as determined by the luciferase assay described herein.

22. A nucleotide sequence encoding a single-chain polypeptide according to claim 1.

23. An expression vector comprising a nucleotide sequence according to claim 22.

24. A recombinant host cell comprising an expression vector according to claim 23.

25. A method for preparing a single-chain multimeric polypeptide having G-CSF activity, comprising culturing a recombinant host cell according to claim 24 comprising a single nucleotide sequence encoding said polypeptide in a suitable culture medium under conditions permitting expression of the nucleotide sequence, and recovering the resulting polypeptide from the cell culture

26. The method of claim 25, further comprising subjecting the polypeptide to conjugation in vitro with a non-polypeptide moiety under suitable reaction conditions to result in a polypeptide conjugate.

27. A composition comprising a single-chain multimeric polypeptide according to claim 1 together with at least one excipient or vehicle.

28. A method of treating a mammal having a general haematopoietic disorder, including disorders arising from chemotherapy, radiation therapy or bone marrow transplantations, treatment of AIDS or other immunodeficiency diseases, treatment of leukopenia and treatment of acute myeloid leukaemia, comprising administering to a mammal in need thereof an effective amount of a single-chain multimeric polypeptide according to claim 1.

29. A single-chain multimeric polypeptide having G-CSF activity, comprising at least two monomeric units independently selected from (a) hG-CSF with the amino acid sequence shown in SEQ ID NO:1 and (b) variants of hG-CSF, said monomeric units being linked via a peptide bond or a peptide linker, wherein the polypeptide has at least one non-polypeptide moiety covalently bound to an attachment group of the polypeptide.

30. The single-chain multimeric polypeptide of claim 29, comprising at least one covalently bound non-polypeptide moiety selected from the group consisting of polymer molecules, lipophilic compounds, oligosaccharide moieties and organic derivatizing agents.

31. A single-chain multimeric polypeptide having G-CSF activity, comprising at least two monomeric units independently selected from (a) hG-CSF with the amino acid sequence shown in SEQ ID NO:1 and (b) variants of hG-CSF, said monomeric units being linked via a peptide bond or a peptide linker, the polypeptide comprising at least one covalently bound polyethylene glycol molecule.

32. The single-chain multimeric polypeptide of claim 31, comprising at least one polyethylene glycol molecule bound to a lysine residue.

33. The single-chain multimeric polypeptide of claim 31, comprising two monomeric units comprising the amino acid sequence of hG-CSF as shown in SEQ ID NO:1.
